(19) United States
(12) Patent Application Publication
(10) Pub. No.: US 2012/0128657 A1
(43) Pub. Date: May 24, 2012

(54) SYNTHETIC MACROCYCLIC COMPOUNDS AND METHODS FOR TREATING CANCER

(76) Inventors: Carole A. Bewley, Bethesda, MD (US); Belhu B. Metaferia, Bethesda, MD (US); Xin-Yu Huang, New York, NY (US)

(21) Appl. No.: 12/526,477

(22) PCT Filed: Feb. 7, 2008
(86) PCT No.: PCT/US08/53334

§ 371 Date: Aug. 7, 2009

Related U.S. Application Data
(60) Provisional application No. 60/900,151, filed on Feb. 7, 2007.

(51) Int. Cl. A61K 31/395; A61K 38/43; A61K 39/395; A61K 31/704; A61K 33/24; A61K 31/422; A61K 31/437; A61K 31/519; A61K 31/57; A61K 38/50; A61P 35/00; A61P 35/02; A61P 35/04; A61P 27/02; A61P 9/10; A61P 37/06; A61P 1/00; A61P 29/00; A61P 7/06; C07D 267/00

(52) U.S. Cl. 424/130.1; 540/456; 514/450; 424/94.1; 514/34; 424/649; 514/374; 514/283; 514/279; 514/249; 514/171; 424/94.6

(57) ABSTRACT

Disclosed herein are macrocyclic compounds that are effective to inhibit cell migration. In one embodiment, the compounds have the structure: or any pharmaceutically acceptable salt or solvate thereof, wherein: m is 0 or 1; R¹, R² and R³ independently are H, aralkyl, acyl, lower alkyl or silyl; X is —C(O)N(R⁴)— or —C(S)N(R⁴)—; —C(O)—; —C(S)—; Y is —OC(O)—; —OC(O)N(R⁵)—; —N(R⁵)C(O)—; or —OC(O)O—; G comprises a saturated or unsaturated aliphatic chain having from 2 to about 10 atoms in the chain, the chain optionally including 1, 2, or 3 heteroatoms; the chain optionally being substituted with 1, 2 or 3 substituents independently selected from lower alkyl, —OR⁶, epoxy, aziridinyl, cyclopropyl, —NR⁷R⁸ and halo; R⁴, R⁵, R⁶, R⁷ and R⁸ independently are selected from H, lower alkyl and acyl. Also disclosed are methods for making and using compounds as well as pharmaceutical compositions including one or more of the disclosed macrocycles.

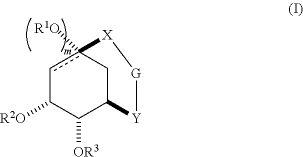

### FIELD

Disclosed herein are synthetic macrocyclic compounds and pharmaceutical compositions as well as methods for using the compounds and compositions.

### BACKGROUND

Abnormal cell migration is implicated in a number of disease states. Of the diseases characterized by abnormal cell migration, cancer is the leading cause of death. Cancer is typically characterized by an increase in the number of abnormal, or neoplastic cells that proliferate to form a tumor mass. Cancer cells must acquire several traits for tumor growth and progression to occur. Tumor cells also must acquire the ability to spread or migrate by invading adjacent tissue and/or metastasizing to distant sites. Moreover, the tumor must generate new vasculature to supply blood to nourish the tumor. This process, referred to as angiogenesis, also involves cell migration.

Cell migration, at least in adults, is relatively rare under normal physiological conditions. Similarly, angiogenesis is a highly regulated process under normal conditions. Nonetheless, in disease states, regulation of cell migration and angiogenesis becomes uncoupled from normal regulatory restrictions, and many diseases are supported by persistent unregulated cell migration and/or angiogenesis. Otherwise stated, unregulated cell migration and angiogenesis may either cause a particular disease directly or exacerbate an existing pathological condition. Accordingly, there is a need for inhibitors of cell migration and angiogenesis.

### SUMMARY

Disclosed herein are novel macrocyclic compounds. In one embodiment, the disclosed compounds are represented by the structure:

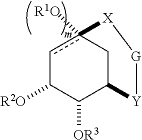

or any pharmaceutically acceptable salt thereof, wherein:

m is 0 or 1;

R¹, R² and R³ independently are H, aralkyl, acyl, lower alkyl or silyl;

X is —C(O)N(R⁴)— or —C(S)N(R⁴)—; —C(O)—; or —C(S)—;

Y is —OC(O)—; —OC(O)N(R⁵)—; —N(R⁵)C(O)—; or —OC(O)O—;

G comprises a saturated or unsaturated aliphatic chain having from 2 to about 10 atoms in the chain, the chain optionally including 1, 2, or 3 heteroatoms; the chain optionally being substituted with 1, 2 or 3 substituents independently selected from lower alkyl, —OR⁶, epoxy, aziridinyl, cyclopropyl, —NR⁷R⁸ and halo;

R⁴, R⁵, R⁶, R⁷ and R⁸ independently are selected from H, lower alkyl and acyl.

Also disclosed herein are methods for making the disclosed macrocyclic compounds. In exemplary embodiments the disclosed methods include a macrolactonization, a macrolactamization or a ring closing metathesis reaction.

Another aspect of the disclosure includes pharmaceutical compositions including the disclosed macrocyclic compounds as well as methods for their use. In one embodiment, disclosed macrocyclic compounds are employed to treat a pathologic condition that is ameliorated by decreasing or inhibiting cell migration. Another method for using the disclosed compounds and compositions includes decreasing anchorage-dependent growth of tumor cells. In one aspect, the methods can be used to treat any pathologic condition characterized by neovascularization. Neovascularization or angiogenesis is associated with a host of different disorders, including neoplasia (such as cancer) ocular neovascular disease, hemangioma, and disorders of chronic inflammation, such as rheumatoid arthritis, Crohn's disease, and many ophthalmic diseases that induce neovascularization of the retina or cornea.

In one embodiment a disclosed method includes treating a subject suspected to have metastatic cancer cells with one or more compounds according to the formula above to decrease or inhibit cell migration, thereby retarding angiogenesis and/or tumor progression. Some examples of the disclosed cell migration inhibitors are not cytotoxic. Thus, in one aspect, the disclosed compounds are co-administered with another chemotherapeutic agent, such as an anticancer agent.

The foregoing and other objects, features, and advantages of the invention will become more apparent from the following detailed description, which proceeds with reference to the accompanying figures.

### BRIEF DESCRIPTION OF THE DRAWINGS

FIG. 1 is a graph illustrating 4T1 cell proliferation, wherein each data point represents the mean±SEM (n=3).

FIG. 2A includes micrographs from an in vitro wound healing assay in the absence (left) or presence (center) of serum, and in the presence of serum plus compound 1 (right).

FIG. 2B includes micrographs from a lemellipodia formation assay using 4T1 breast cancer cells in the absence (left) or presence (center) of serum, and in the presence of serum plus compound 1 (right).

FIG. 3 includes ROESY spectra for compounds 1 and 2 recorded in CD₃OD at 600 MHz and schematics indicating dipolar couplings observed for each compound, which confirm E and Z stereochemistry for 1 and 2, respectively.

### DETAILED DESCRIPTION

The following explanations of terms and methods are provided to better describe the present compounds, compositions and methods, and to guide those of ordinary skill in the art in the practice of the present disclosure. It is also to be understood that the terminology used in the disclosure is for the purpose of describing particular embodiments and examples only and is not intended to be limiting.

As used herein, the singular terms “a,” “an,” and “the” include plural referents unless context clearly indicates otherwise. Similarly, the word “or” is intended to include “and” unless the context clearly indicates otherwise. Also, as used herein, the term “comprises” means “includes.” Hence “comprising A or B” means including A, B, or A and B.

Variables such as R¹, R², R³, R⁴, R⁵, R⁶, R⁷, R⁸, R⁹, R¹⁰, R¹¹, G, J, m, n, X, Y and Z, used throughout the disclosure are the same variables as previously defined unless stated to the contrary.

“Optional” or “optionally” means that the subsequently described event or circumstance can but need not occur, and that the description includes instances where said event or circumstance occurs and instances where it does not.

“Derivative” refers to a compound or portion of a compound that is derived from or is theoretically derivable from a parent compound.

The term “subject” includes both human and veterinary subjects.

“Treatment” refers to a therapeutic intervention that ameliorates a sign or symptom of a disease or pathological condition after it has begun to develop. As used herein, the term “ameliorating,” with reference to a disease or pathological condition, refers to any observable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, an improvement in the overall health or well-being of the subject, or by other parameters well known in the art that are specific to the particular disease. The phrase “treating a disease” refers to inhibiting the full development of a disease or condition, for example, in a subject who is at risk for a disease such as cancer, particularly a metastatic cancer. A “prophylactic” treatment is a treatment administered to a subject who does not exhibit signs of a disease or exhibits only early signs for the purpose of decreasing the risk of developing pathology. By the term “coadminister” is meant that each of at least two compounds be administered during a time frame wherein the respective periods of biological activity overlap. Thus, the term includes sequential as well as coextensive administration of two or more drug compounds.

The term “neoplasm” refers to an abnormal cellular proliferation, which includes benign and malignant tumors, as well as other proliferative disorders.

The term “acyl” refers group of the formula RC(O)— wherein R is an organic group.

The term “alkyl” refers to a branched or unbranched saturated hydrocarbon group of 1 to 24 carbon atoms, such as methyl, ethyl, n-propyl, isopropyl, n-butyl, isobutyl, t-butyl, pentyl, hexyl, heptyl, octyl, decyl, tetradecyl, hexadecyl, eicosyl, tetracosyl and the like. A “lower alkyl” group is a saturated branched or unbranched hydrocarbon having from 1 to 10 carbon atoms.

The term “alkenyl” refers to a hydrocarbon group of 2 to 24 carbon atoms and structural formula containing at least one carbon-carbon double bond.

The term “alkynyl” refers to a hydrocarbon group of 2 to 24 carbon atoms and a structural formula containing at least one carbon-carbon triple bond.

The terms “halogenated alkyl” or “haloalkyl group” refer to an alkyl group as defined above with one or more hydrogen atoms present on these groups substituted with a halogen (F, Cl, Br, I).

The term “cycloalkyl” refers to a non-aromatic carbon-based ring composed of at least three carbon atoms. Examples of cycloalkyl groups include, but are not limited to, cyclopropyl, cyclobutyl, cyclopentyl, cyclohexyl, and the like. The term “heterocycloalkyl group” is a cycloalkyl group as defined above where at least one of the carbon atoms of the ring is substituted with a heteroatom such as, but not limited to, nitrogen, oxygen, sulfur, or phosphorous.

The term “aliphatic” is defined as including alkyl, alkenyl, alkynyl, halogenated alkyl and cycloalkyl groups as described above. A “lower aliphatic” group is a branched or unbranched aliphatic group having from 1 to 10 carbon atoms.

The term “aryl” refers to any carbon-based aromatic group including, but not limited to, benzene, naphthalene, etc. The term “aromatic” also includes “heteroaryl group,” which is defined as an aromatic group that has at least one heteroatom incorporated within the ring of the aromatic group. Examples of heteroatoms include, but are not limited to, nitrogen, oxygen, sulfur, and phosphorous. The aryl group can be substituted with one or more groups including, but not limited to, alkyl, alkynyl, alkenyl, aryl, halide, nitro, amino, ester, ketone, aldehyde, hydroxy, carboxylic acid, or alkoxy, or the aryl group can be unsubstituted. The term “alkyl amino” refers to alkyl groups as defined above where at least one hydrogen atom is replaced with an amino group.

“Carbonyl” refers to a radical of the formula —C(O)—. Carbonyl-containing groups include any substituent containing a carbon-oxygen double bond (C═O), including acyl groups, amides, carboxy groups, esters, ureas, carbamates, carbonates and ketones and aldehydes, such as substituents based on —COR or —RCHO where R is an aliphatic, heteroaliphatic, alkyl, heteroalkyl, hydroxyl, or a secondary, tertiary, or quaternary amine.

“Carboxyl” refers to a —COOH radical. Substituted carboxyl refers to —COOR where R is aliphatic, heteroaliphatic, alkyl, heteroalkyl, or a carboxylic acid or ester.

The term “hydroxyl” is represented by the formula —OH. The term “alkoxy group” is represented by the formula —OR, where R can be an alkyl group, optionally substituted with an alkenyl, alkynyl, aryl, aralkyl, cycloalkyl, halogenated alkyl, or heterocycloalkyl group as described above.

“Epoxy” or “epoxide” refers to a cyclic ether having three atoms; similarly “aziridinyl” refers to a cyclic amine having two carbon atoms bonded to the nitrogen in a three-membered ring. A moiety that is said to be substituted with an epoxy group, contains two carbons bonded to one another and to the same oxygen atom. Likewise, a moiety substituted with an aziridinyl group contains two carbon atoms bonded to one another and to the same nitrogen atom. In such compounds the aziridinyl nitrogen may be optionally substituted. “Cyclopropyl” refers to an alicyclic three-membered ring. Cyclopropyl groups too may be optionally substituted.

The term “hydroxyalkyl” refers to an alkyl group that has at least one hydrogen atom substituted with a hydroxyl group. The term “alkoxyalkyl group” is defined as an alkyl group that has at least one hydrogen atom substituted with an alkoxy group described above.

The term “amine” or “amino” refers to a group of the formula —NRR′, where R and R′ can be, independently, hydrogen or an alkyl, alkenyl, alkynyl, aryl, aralkyl, cycloalkyl, halogenated alkyl, or heterocycloalkyl group described above.

The term “amide group” is represented by the formula —C(O)NRR′, where R and R′ independently can be a hydrogen, alkyl, alkenyl, alkynyl, aryl, aralkyl, cycloalkyl, halogenated alkyl, or heterocycloalkyl group described above.

The term “aralkyl” refers to an aryl group having an alkyl group, as defined above, attached to the aryl group. An example of an aralkyl group is a benzyl group.

Optionally substituted groups, such as “optionally substituted alkyl,” refers to groups, such as an alkyl group, that when substituted, have from 1-5 substituents, typically 1, 2 or 3 substituents, selected from alkoxy, optionally substituted alkoxy, acyl, acylamino, acyloxy, amino, aminoacyl, aminoacyloxy, aryl, carboxyalkyl, optionally substituted cycloalkyl, optionally substituted cycloalkenyl, halogen, optionally substituted heteroaryl, optionally substituted heterocyclyl, hydroxy, sulfonyl, thiol and thioalkoxy. In particular, optionally substituted alkyl groups include, by way of example, haloalkyl groups, such as fluoroalkyl groups, including, without limitation, trifluoromethyl groups.

The term “prodrug” also is intended to include any covalently bonded carriers that release a disclosed compound or a parent thereof in vivo when the prodrug is administered to a subject. Since prodrugs often have enhanced properties relative to the active agent pharmaceutical, such as, solubility and bioavailability, the compounds disclosed herein can be delivered in prodrug form. Thus, also contemplated are prodrugs of the presently claimed compounds, methods of delivering prodrugs and compositions containing such prodrugs. Prodrugs of the disclosed compounds typically are prepared by modifying one or more functional groups present in the compound in such a way that the modifications are cleaved, either in routine manipulation or in vivo, to yield the parent compound. In particular, ester prodrugs are specifically contemplated herein. For example, one or more hydroxyl functions on the quinic or shikimic acid-derived portion of the disclosed macrocycles may be masked with any group that is cleaved to yield the corresponding hydroxyl, such as an acyl group. Similarly, prodrugs include compounds having an amino or sulfhydryl group functionalized with any group that is cleaved to yield the corresponding free amino or free sulfhydryl group. Examples of prodrugs include, without limitation, compounds having a hydroxy, amino and/or sulfhydryl group acylated with an acetate, formate, or benzoate group.

Protected derivatives of the disclosed compound also are contemplated. The term “protecting group” or “blocking group” refers to any group that when bound to a functional group prevents or diminishes the group's susceptibility to reaction. “Protecting group” generally refers to groups well known in the art which are used to prevent selected reactive groups, such as carboxy, amino, hydroxy, mercapto and the like, from undergoing undesired reactions, such as nucleophilic, electrophilic, oxidation, reduction and the like. The terms “deprotecting,” “deprotected,” or “deprotect,” as used herein, are meant to refer to the process of removing a protecting group from a compound. Preferred protecting groups are indicated herein where appropriate. Examples of hydroxy protecting groups include, but are not limited to, aralkyl, substituted aralkyl, cycloalkenylalkyl and substituted cycloalkenyl alkyl, allyl, substituted allyl, acyl, alkoxycarbonyl, aralkoxycarbonyl, silyl groups and the like. Examples of aralkyl protecting groups include, but are not limited to, benzyl, ortho-methylbenzyl, trityl and benzhydryl, which can be optionally substituted with halogen, alkyl, alkoxy, hydroxy, nitro, acylamino, acyl and the like, and salts, such as phosphonium and ammonium salts. Examples of aryl groups include phenyl, naphthyl and the like. Suitable acyl, alkoxycarbonyl and aralkoxycarbonyl groups include benzyloxycarbonyl, t-butoxycarbonyl, iso-butoxycarbonyl, benzoyl, substituted benzoyl, butyryl, acetyl, trifluoroacetyl, trichloroacetyl and the like. Many of the hydroxy protecting groups are also suitable for protecting amino, carboxy and mercapto groups. For example, aralkyl groups can be used to protect all of these functionalities. Alkyl groups are also suitable groups for protecting, in addition to hydroxy groups, carboxy and mercapto groups, for example, tert-butyl groups can be used to mask these functionalities.

Neighboring functional groups can be protected using the same protecting group. For example, diols, such as vicinal diols can be masked as an acetal moiety, produced by their condensation with, by way of example, a ketone. A particularly useful acetal for masking vicinal hydroxyl groups is an acetonide, which is produced, for example, via the condensation of the diol with acetone.

Silyl protecting groups or silyl groups are silicon atoms optionally substituted by one or more alkyl, aryl and/or aralkyl groups. Suitable silyl protecting groups include, but are not limited to, trimethylsilyl, triethylsilyl, triisopropylsilyl, tert-butyldimethylsilyl, dimethylphenylsilyl, 1,2-bis(dimethylsilyl)benzene, 1,2-bis(dimethylsilyl)ethane and diphenylmethylsilyl. Silylation of amino groups, for example, provides mono- or di-silylamino groups. Removal of the silyl function from a silyl ether or amine function is readily accomplished by treatment with, for example, a metal hydroxide or ammonium fluoride reagent, either as a discrete reaction step or in situ during a reaction with the alcohol group. Suitable silylating agents are, for example, silyl halides or sulfonates, such as trimethylsilyl chloride, tert-butyl-dimethylsilyl chloride, tert-butyl-dimethylsilyl bromide, tert-butyl-dimethylsilyl triflate, phenyldimethylsilyl chloride, diphenylmethyl silyl chloride or their combination products with imidazole or DMF. Methods for silylation of hydroxy groups and amino groups are well known to those skilled in the art as is the removal of silyl protecting groups. Methods of preparation of hydroxy and/or amino protected derivatives, as well as their deprotection is well known to those skilled in the art of organic chemistry. The chemical properties of the above listed protecting groups as well as others, along with methods for their introduction and their removal are described, for example, in Greene and Wuts, Protective Groups in Organic Synthesis (3rd ed.), John Wiley & Sons, NY (1999).

In general, protecting groups are removed under conditions which will not affect the remaining portion of the molecule. These methods are well known in the art and include acid hydrolysis, hydrogenolysis and the like. A preferred method involves removal of a protecting group, such as removal of a benzyl group by hydrogenolysis utilizing palladium on carbon in a suitable solvent system such as an alcohol, acetic acid, and the like or mixtures thereof. A t-butoxycarbonyl protecting group can be removed utilizing an inorganic or organic acid, such as HCl or trifluoroacetic acid, in a suitable solvent system, such as dioxane or methylene chloride. The resulting amino salt can readily be neutralized to yield the free amine. Similarly, acetal-based protecting groups, such as acetonides can be cleaved under acid-catalyzed hydrolysis conditions. Carboxy protecting group, such as methyl, ethyl, benzyl, tert-butyl, 4-methoxyphenylmethyl and the like, can be removed under hydrolysis and hydrogenolysis conditions well known to those skilled in the art.

“Saturated or unsaturated” includes substituents saturated with hydrogens, substituents completely unsaturated with hydrogens and substituents partially saturated with hydrogens.

“Solvate” refers to a compound physically associated with one or more solvent molecules. This physical association involves varying degrees of ionic and covalent bonding, including by way of example covalent adducts and hydrogen bonded solvates. In certain instances the solvate will be capable of isolation, for example when one or more solvent molecules are incorporated in the crystal lattice of the crystalline solid. “Solvate” encompasses both solution-phase and isolable solvates. Representative solvates include ethanol associated compounds, methanol associated compounds, and the like. “Hydrate” is a solvate wherein the solvent molecule(s) is/are H₂O.

“Sulfonyl” refers to a group —S(O)₂R, wherein R is an organic group, such as an alkyl, aralkyl or aryl group.

In general compounds are referred to herein by common and IUPAC naming conventions as will be understood by those of ordinary skill in the art. Certain compounds, such as natural products, typically referred to by their common names. For example, the IUPAC name for quinic acid is 1R,3R,4S,5R-tetrahydroxycyclohexanecarboxylic acid, but will be referred to herein as quinic acid. Similarly, shikimic acid is referred to herein by its common name.

It is understood that substituents and substitution patterns of the compounds described herein can be selected by one of ordinary skill in the art to provide compounds that are chemically stable and that can be readily synthesized by techniques known in the art and further by the methods set forth in this disclosure. Reference will now be made in detail to the present preferred embodiments.

### I. Macrocyclic Compounds

Historically, natural products have accounted for the majority of drugs and drug leads. In contrast, disclosed herein are novel drug compounds designed without using a natural product as a template. More particularly the disclosed compounds are novel macrocyclic compounds. Examples of the disclosed macrocycles have been found to have, inter alia, efficacy as inhibitors of cell migration.

Exemplary compounds disclosed herein have the structure:

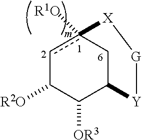

or any pharmaceutically acceptable salt or solvate thereof, wherein:

m is 0 or 1;

R¹, R² and R³ independently are H, aralkyl, acyl, lower alkyl or silyl;

X is —C(O)N(R⁴)— or —C(S)N(R⁴)—; —C(O)—; or —C(S)—;

Y is —OC(O)—; —OC(O)N(R⁵)—; —N(R⁵)C(O)—; or —OC(O)O—;

G comprises a saturated or unsaturated aliphatic chain having from 2 to about 10 atoms in the chain, the chain optionally including 1, 2, or 3 heteroatoms; the chain optionally being substituted with 1, 2 or 3 substituents independently selected from lower alkyl, —OR⁶, epoxy, aziridinyl, cyclopropyl, —NR⁷R⁸ and halo;

R⁴, R⁵, R⁶, R⁷ and R⁸ independently are selected from H, lower alkyl and acyl.

With reference to the formula above, when m is 0, the compound has a double bond between positions 1 and 2 and has the structure

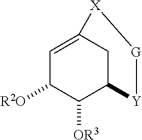

Compounds wherein m is 1 are represented by the structure

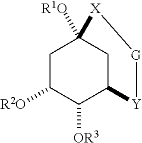

With reference to the formulas above, in particular embodiments G includes an optionally substituted unsaturated aliphatic chain. When G includes such an aliphatic chain, it typically includes an alkenyl moiety in the chain. Such alkenyl groups can be E or Z stereoisomers. Whether G is saturated or unsaturated, G can include from 2 to about 10, such as from 4 to 8 carbon atoms in the aliphatic chain. G typically includes 4, 5 or 6 carbon atoms in the chain. This count of carbon atoms does not include carbon atoms in optional substituents on the chain. Thus, when G is substituted, the group G can include more than the 2 to about 10 or 4 to 8 atoms present in the chain. In particular embodiments, G comprises a group J in the optionally substituted aliphatic chain, wherein J has the structure

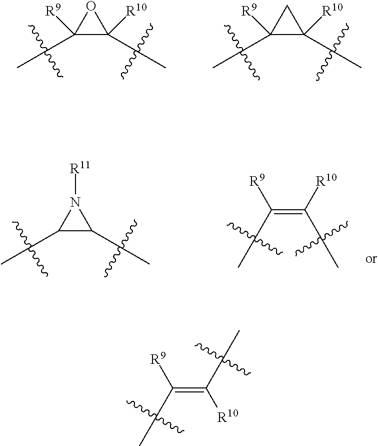

With reference to the structures for J, R⁹ and R¹⁰ independently are H or optionally substituted lower alkyl, such as methyl, ethyl, propyl and the like; and R¹¹ is H, lower alkyl, acyl or sulfonyl.

In certain embodiments, X is a carbonyl functional group and Y is an oxycarbonyl moiety. When Y is an ester group, such compounds are referred to as macrolides. Examples of such compounds include those of the structure

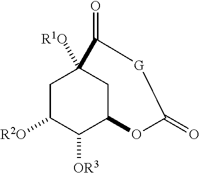

In one aspect, the disclosed macrolides include those wherein X includes an optionally substituted amide group. Certain such compounds have the formula:

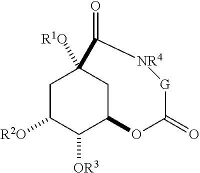

When X includes a substituted amide, such as when R⁴ is not H, R⁴ can be lower alkyl, such as methyl, or acyl. In certain embodiments, at least one of R¹, R² and R³ is an acyl group. For example, when R¹, R² and/or R³ are hydroxy groups, they can be esterified, optionally selectively, to provide such acyl compounds. Exemplary acyl groups include aromatic and lower aliphatic acyl groups, including alkyl acyl groups, such as acetyl and haloalkyl acyl groups, such as trifluoracetyl. Additional suitable acyl groups include unsaturated aliphatic acyl groups, such as lower aliphatic groups, including by way of example 2-butenoate esters. Other acyl groups include aryl and aralkyl acyl groups, such as benzoyl groups. Other acyl groups include amino acid esters, including protected and unprotected amino acid esters.

In certain embodiments the compounds include one or more methylene groups in the macrocycle. Examples of such compounds have the formula

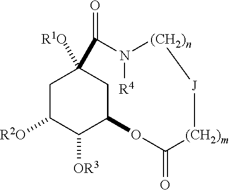

wherein n is an integer from 1 to 5; and

m is an integer from 0 to 5.

In certain embodiments, the compounds have the formula

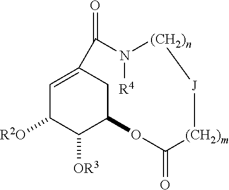

Typically, m and n independently are 1, 2 or 3. For example, certain compounds having m equal 2 and n equal 1 can be represented by the formula

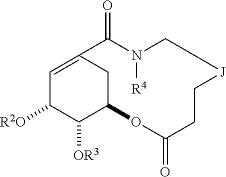

Certain compounds having the formulas above can be represented by the formula

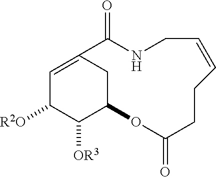

Typically, R¹, R² and R³ in the formulas above represent H or a group that is removed to unmask the corresponding free hydroxyl group. For example, in prodrug compounds R¹, R² and/or R³ may be an acyl group that is cleaved under physiological conditions to provide the free hydroxyl compound in vivo. Typically, in the final product, R¹, R² and R³ each are H.

Particular examples of macrolide compounds disclosed herein include

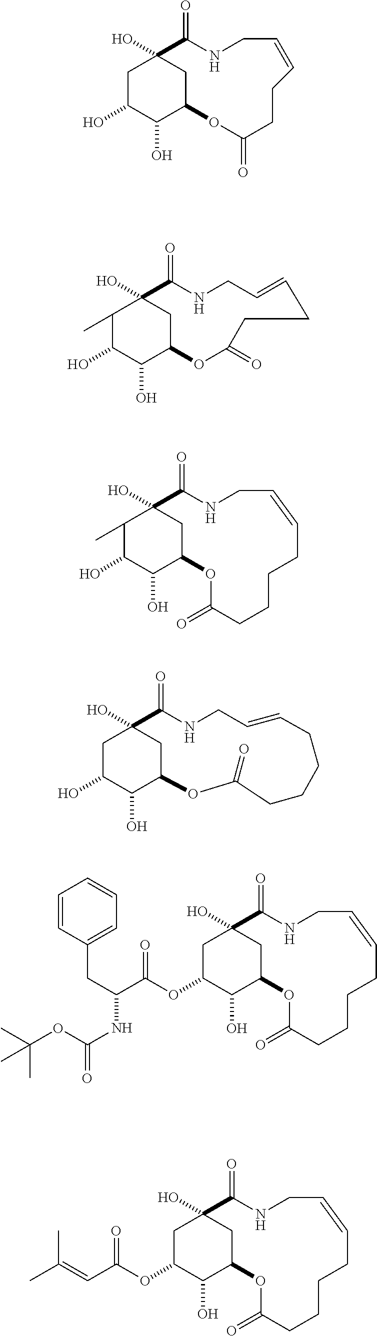

The foregoing compounds include several asymmetric centers; thus these compounds can exist in various stereoisomeric forms, including enantiomers and diastereomers. Thus, compounds and compositions may be provided as individual pure enantiomers, diastereomers or geometric isomers, or as stereoisomeric mixtures. In certain embodiments the compounds disclosed herein are provided in substantially enantiopure form, such as in a 90% enantiomeric excess, a 95% enantiomeric excess, a 97% enantiomeric excess or even in greater than a 99% enantiomeric excess, such as in enantiopure form. Similarly, certain compounds disclosed herein include one or more double bonds that can exist as E or Z isomers. Unless otherwise indicated, both E and Z isomers are included in the description of such double-bond-containing compounds.

### II. Methods for Synthesis

Exemplary methods for making the disclosed macrolide compounds are disclosed herein and additional methods for making the compounds will be apparent to those of skill in the art of organic synthesis upon consideration of the present specification.

One approach to the synthesis of the disclosed macrocycles employs a macrolactamization reaction to close the macrocyclic ring. Reagents and conditions for performing such amide bond forming reactions are well known to those of skill in the art. One example of this approach includes providing an intermediate compound of the structure

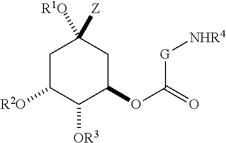

wherein Z is —C(O)OH or an activated ester moiety; and

performing a macrolactamization reaction to provide the macrocycle. More specifically the macrolactam precursor compound can have the quinic acid-derived structure:

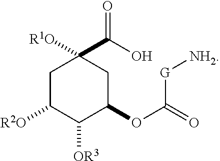

Alternatively, the macrocycle can be produced by a macrolactonization reaction. Conditions and reagents, such as Yamaguchi conditions, for macrolactonization are well known to those of skill in the art of organic synthesis. One macrolactonization-based synthetic approach involves an intermediate having the structure

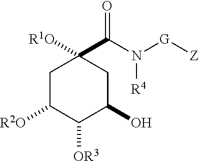

wherein Z is —C(O)OH or an activated ester moiety; and cyclizing this compound via an ester-bond forming reaction to produce a macrolide compound. Such an intermediate also could have the structure:

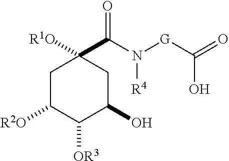

In another embodiment of a synthesis of the disclosed macrocyclic compounds a ring closing metathesis reaction is used to close a macrocyclic ring of the compound. In recent years, with the development of new, well-defined, functional group-tolerant metathesis catalysts, metathesis chemistry has been applied to polymer chemistry and complex total syntheses. See, for example, Fürstner, A. Olefin Metathesis and Beyond. Angew. Chem., Int. Ed. Engl. 2000, 39, 3012-3043.

In principle, any known or future-developed metathesis catalyst may be used in accordance with embodiments of the present macrocyclization method. Typical metathesis catalysts used for disclosed embodiments include metal carbene catalysts based upon transition metals, such as ruthenium and molybdenum. Exemplary ruthenium-based metathesis catalysts include those represented by structures A, B and C.

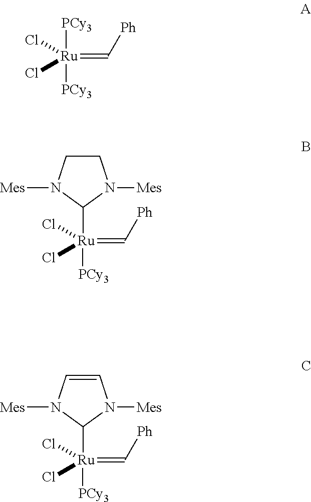

Additional metathesis catalysts include, without limitation, metal carbene complexes selected from the group consisting of molybdenum, osmium, chromium, rhenium, tungsten and tungsten carbene complexes. The term “complex” refers to a metal atom, such as a transition metal atom, with at least one ligand or complexing agent coordinated or bound thereto. Such a ligand typically is a Lewis base in metal carbene complexes useful for alkene, alkyne or alkene-metathesis. Typical examples of such ligands include phosphines, halides and stabilized carbenes. Some metathesis catalysts employ plural metals or metal co-catalysts. For example, German patent publication No. A1-282594, which is incorporated herein by reference, discloses a catalyst comprising a tungsten halide, a tetraalkyl tin compound, and an organoaluminum compound.

An immobilized catalyst can be used for the metathesis process. See, for example, Blechert, et al. Synthesis and Application of a Permanently Immobilized Olefin Metathesis Catalyst. Angew. Chem. Int. Ed. Engl. 2000, 39, 3898-3901, incorporated herein by reference. Such an immobilized catalyst can be used in a flow process as is known to those of ordinary skill in the art. An immobilized catalyst can simplify purification of products and recovery of the catalyst, so that recycling the catalyst is convenient.

One example of a ring closing metathesis-based approach to the disclosed macrocycles involves subjecting an intermediate compound of the formula

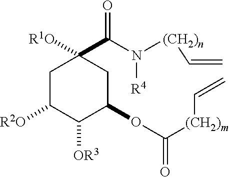

to ring closing metathesis conditions, such as by contacting the intermediate compound with a metathesis catalyst. Typically the metathesis catalyst selected is ruthenium or molybdenum-based catalyst. This ring closing reaction usually generates a mixture of E and Z alkene stereoisomers, which may be separated, such as by chromatographic means. In one embodiment, ring closing metathesis products are subjected to hydrogenation, typically with a noble metal catalyst, such as palladium on carbon, to reduce the alkene moiety produced by the metathesis reaction. Such hydrogenation reactions can be performed on mixtures of alkene isomers to produce a single product.

### III. Pharmaceutical Compositions and Methods for their Use

Another aspect of the disclosure includes pharmaceutical compositions prepared for administration to a subject and which include a therapeutically effective amount of one or more of the currently disclosed compounds. The therapeutically effective amount of a disclosed compound will depend on the route of administration, the species of subject and the physical characteristics of the subject being treated. Specific factors that can be taken into account include disease severity and stage, weight, diet and concurrent medications. The relationship of these factors to determining a therapeutically effective amount of the disclosed compounds is understood by those of skill in the art.

Pharmaceutical compositions for administration to a subject can include carriers, thickeners, diluents, buffers, preservatives, surface active agents and the like in addition to the molecule of choice. Pharmaceutical compositions can also include one or more additional active ingredients such as antimicrobial agents, anti-inflammatory agents, anesthetics, and the like. Pharmaceutical formulations can include additional components, such as carriers. The pharmaceutically acceptable carriers useful for these formulations are conventional. Remington's Pharmaceutical Sciences, by E. W. Martin, Mack Publishing Co., Easton, Pa., 19th Edition (1995), describes compositions and formulations suitable for pharmaceutical delivery of the compounds herein disclosed.

In general, the nature of the carrier will depend on the particular mode of administration being employed. For instance, parenteral formulations usually contain injectable fluids that include pharmaceutically and physiologically acceptable fluids such as water, physiological saline, balanced salt solutions, aqueous dextrose, glycerol or the like as a vehicle. For solid compositions (for example, powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate. In one aspect, disclosed methods include treating ocular disorders, such as ocular neovascular disease. Accordingly, embodiments of pharmaceutical compositions include those formulated for ophthalmic administration, such as in an eye drop or an intraocular implant.

In one embodiment, the disclosed compounds are used in conjunction with a stent to inhibit reocclusion or restenosis of, for example, a blood vessel, particularly a coronary artery. Stents are commonly used to open blood vessels, clear obstructions, prevent restenosis, provide support at the site of an aneurysm, and to repair damage to vascular tissues. In addition to blood vessels, other vessels of the body may be repaired with stents, including the trachea for breathing disorders, renal and urethral tubules, fallopian tubes for the treatment of infertility and eustachian tubes for the treatment of chronic ear infection and other hearing disorders.

Many stent designs are known and are in clinical use. Stents can be, for example, cut from a tube or formed from a wire that has been bent back and forth in a zig-zag pattern and wound in a circumferential direction to form one or more loops of a pre-determined circumference. Typically, the stent is radially expandable from a collapsed condition. Once in position, it is expanded to the predetermined size, to support and reinforce the lumen. The present compounds can be delivered by such stents, drug eluting stents, for the inhibition of cell migration and angiogenesis associated, for example with restenosis. The disclosed macrocycles can be delivered, as is known to those of skill in the art by stents such as those described in U.S. Pat. No. 6,004,346; U.S. Pat. No. 5,972,027; U.S. Pat. No. 5,697,967 and/or U.S. Pat. No. 6,335,029. Each of these patents is incorporated herein by reference.

Pharmaceutical compositions disclosed herein include those formed from pharmaceutically acceptable salts and/or solvates of the disclosed compounds. Pharmaceutically acceptable salts include those derived from pharmaceutically acceptable inorganic or organic bases and acids. Particular disclosed compounds possess at least one basic group that can form acid-base salts with acids. Examples of basic groups include, but are not limited to, amino and imino groups. Examples of inorganic acids that can form salts with such basic groups include, but are not limited to, mineral acids such as hydrochloric acid, hydrobromic acid, sulfuric acid or phosphoric acid. Basic groups also can form salts with organic carboxylic acids, sulfonic acids, sulfo acids or phospho acids or N-substituted sulfamic acid, for example acetic acid, propionic acid, glycolic acid, succinic acid, maleic acid, hydroxymaleic acid, methylmaleic acid, fumaric acid, malic acid, tartaric acid, gluconic acid, glucaric acid, glucuronic acid, citric acid, benzoic acid, cinnamic acid, mandelic acid, salicylic acid, 4-aminosalicylic acid, 2-phenoxybenzoic acid, 2-acetoxybenzoic acid, embonic acid, nicotinic acid or isonicotinic acid, and, in addition, with amino acids, for example with α-amino acids, and also with methanesulfonic acid, ethanesulfonic acid, 2-hydroxymethanesulfonic acid, ethane-1,2-disulfonic acid, benzenedisulfonic acid, 4-methylbenzenesulfonic acid, naphthalene-2-sulfonic acid, 2- or 3-phosphoglycerate, glucose-6-phosphate or N-cyclohexylsulfamic acid (with formation of the cyclamates) or with other acidic organic compounds, such as ascorbic acid. In particular, suitable salts include those derived from alkali metals such as potassium and sodium, alkaline earth metals such as calcium and magnesium, among numerous other acids well known in the pharmaceutical art.

Certain compounds include at least one acidic group that can form an acid-base salts with an inorganic or organic base. Examples of salts formed from inorganic bases include salts of the presently disclosed compounds with alkali metals such as potassium and sodium, alkaline earth metals, including calcium and magnesium and the like. Similarly, salts of acidic compounds with an organic base, such as an amine (as used herein terms that refer to amines should be understood to include their conjugate acids unless the context clearly indicates that the free amine is intended) are contemplated, including salts formed with basic amino acids, aliphatic amines, heterocyclic amines, aromatic amines, pyridines, guanidines and amidines. Of the aliphatic amines, the acyclic aliphatic amines, and cyclic and acyclic di- and tri-alkyl amines are particularly suitable for use in the disclosed compounds. In addition, quaternary ammonium counterions also can be used.

Particular examples of suitable amine bases (and their corresponding ammonium ions) for use in the present compounds include, without limitation, pyridine, N,N-dimethylaminopyridine, diazabicyclononane, diazabicycloundecene, N-methyl-N-ethylamine, diethylamine, triethylamine, diisopropylethylamine, mono-, bis- or tris-(2-hydroxyethyl)amine, 2-hydroxy-tert-butylamine, tris(hydroxymethyl)methylamine, N,N-dimethyl-N-(2-hydroxyethyl)amine, tri-(2-hydroxyethyl)amine and N-methyl-D-glucamine. For additional examples of “pharmacologically acceptable salts,” see Berge et al., J. Pharm. Sci. 66:1 (1977).

Compounds disclosed herein can be crystallized and can be provided in a single crystalline form or as a combination of different crystal polymorphs. As such, the compounds can be provided in one or more physical form, such as different crystal forms, crystalline, liquid crystalline or non-crystalline (amorphous) forms. Such different physical forms of the compounds can be prepared using, for example different solvents or different mixtures of solvents for recrystallization. Alternatively or additionally, different polymorphs can be prepared, for example, by performing recrystallizations at different temperatures and/or by altering cooling rates during recrystallization. The presence of polymorphs can be determined by X-ray crystallography, or in some cases by another spectroscopic technique, such as solid phase NMR spectroscopy, IR spectroscopy, or by differential scanning calorimetry.

The disclosed compounds can be used to inhibit cell migration in vitro and in vivo and thus can be used in the prevention (including inhibition) or treatment of conditions characterized by abnormal or pathological cell migration activity. For example, the compositions are useful for prevention or treatment of conditions such as tumor metastasis. In certain embodiments the compounds and compositions are used to treat disorders characterized by pathological neovascularization or angiogenesis. Cell migration and angiogenesis are important in more than one stage of tumor metastasis. For example, vascularization of the tumor allows tumor cells to enter circulation in the blood stream. Angiogenesis must occur after tumor cells have left the primary site. In one aspect of a method for treating a neoplasm, the disclosed compounds and compositions are used to decrease the anchorage-dependent growth of tumor cells.

One aspect of the present disclosure includes methods for treating a hyperproliferative disorder by administering a therapeutically effective amount of the disclosed compounds to a subject in need thereof. For example, particular proliferative disorders that can be so treated include solid tumors, such as sarcomas and carcinomas, include fibrosarcoma, myxosarcoma, liposarcoma, chondrosarcoma, osteogenic sarcoma, and other sarcomas, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, bladder carcinoma, and CNS tumors (such as a glioma, astrocytoma, medulloblastoma, craniopharyogioma, ependymoma, pinealoma, hemangioblastoma, acoustic neuroma, oligodendroglioma, menangioma, melanoma, neuroblastoma and retinoblastoma).

The present disclosure also provides methods to treat hyperproliferative disorders that are characterized by metastasis. By way of example, the presently disclosed compounds and compositions can be used to inhibit the metastasis of prostate, breast, colon, bladder, cervical, skin, testicular, kidney, ovarian, stomach, brain, liver, pancreatic or esophageal cancer, or lymphoma, leukemia or multiple myeloma. In certain embodiments the disclosed compounds and compositions are used to treat a subject is at risk of developing a metastatic proliferative disorder.

Examples of hematological tumors that can be treated as disclosed herein include leukemias, including acute leukemias (such as acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia and myelodysplasia.

Also disclosed herein are methods for treating disorders characterized by uncontrolled or persistent and unregulated angiogenesis. Abnormal angiogenesis occurs in a multiplicity of disorders, which are grouped herein as angiogenesis-dependent or angiogenesis-associated disorders.

Certain angiogenesis-dependent disorders are cardiovascular disorders. Examples such conditions that can be treated using the presently disclosed compounds and compositions include, without limitation, polyarteritis, sickle cell anemia, atherosclerosis, artery occlusion and vein occlusion.

Examples of angiogenesis-dependent disorders include tumor metastasis and abnormal growth. In these conditions angiogenesis supports the pathological growth observed in these conditions.

Another example of a disorder involving pathological angiogenesis is ocular neovascular disease. This category of disease is characterized by unwanted invasion of new blood vessels into the structures of the eye, such as the cornea or retina. Age related macular degeneration also involves angiogenic damage, wherein ingrowth of chorioidal capillaries causes visual problems. Neovascularization also is associated with diabetic retinopathy, corneal graft rejection and neovascular glaucoma.

Another disorder that can be that can be treated with the presently disclosed compounds and compositions is rheumatoid arthritis. Angiogenesis has been implicated in rheumatoid arthritis, for example in the neovascularization of the synovial lining of the afflicted joints.

The therapeutically effective amount of the compound or compounds administered can vary depending upon the desired effects and the factors noted above. Typically, dosages will be between about 0.01 mg/kg and 250 mg/kg of the subject's body weight, and more typically between about 0.05 mg/kg and 100 mg/kg, such as from about 0.2 to about 80 mg/kg, from about 5 to about 40 mg/kg, from about 10 to about 30 mg/kg of the subject's body weight, from about 0.1 mg/kg and about 100 mg/kg, such as between about 1.0 mg/kg and about 20 mg/kg. Thus, unit dosage forms can be formulated based upon the suitable ranges recited above and a subject's body weight. The term “unit dosage form” as used herein refers to a physically discrete unit of therapeutic agent appropriate for the subject to be treated.

Alternatively, dosages are calculated based on body surface area and from about 1 mg/m² to about 200 mg/m², such as from about 5 mg/m² to about 100 mg/m² will be administered to the subject per day. In particular embodiments administration of the therapeutically effective amount of the compound or compounds comprises administering to the subject from about 5 mg/m² to about 50 mg/m², such as from about 10 mg/m² to about 40 mg/m² per day. It is currently believed that a single dosage of the compound or compounds is suitable, however a therapeutically effective dosage can be supplied over an extended period of time or in multiple doses per day. Thus, unit dosage forms also can be calculated using a subject's body surface area based on the suitable ranges recited above and the desired dosing schedule.

It is contemplated that in some embodiments the disclosed compounds are used in combination with other types of treatments, such as cancer treatments. For example the disclosed inhibitors may be used with other chemotherapies, including those employing an anti-proliferative agent, such as, without limitation, microtubule binding agent, a toxin, a DNA intercalator or cross-linker, a DNA synthesis inhibitor, a DNA and/or RNA transcription inhibitor, an enzyme inhibitor, a gene regulator, enediyne antibiotics and/or an angiogenesis inhibitor. Additionally, the disclosed compounds can be used in combination with radiation therapy, surgery, or other modalities of cancer therapy.

“Microtubule binding agent” refers to an agent that interacts with tubulin to stabilize or destabilize microtubule formation thereby inhibiting cell division. Examples of microtubule binding agents that can be used in conjunction with the presently disclosed compounds include, without limitation, paclitaxel, docetaxel, vinblastine, vindesine, vinorelbine (navelbine), the epothilones, colchicine, dolastatin 15, nocodazole, podophyllotoxin and rhizoxin. Analogs and derivatives of such compounds also can be used and will be known to those of ordinary skill in the art. For example, suitable epothilones and epothilone analogs for incorporation into the present compounds are described in International Publication No. WO 2004/018478, which is incorporated herein by reference. Taxoids, such as paclitaxel and docetaxel are currently believed to be particularly useful as therapeutic agents in the presently disclosed compounds. In one embodiment, metastatic breast cancer is treated by administering a presently disclosed cell migration inhibitor in combination with a taxoid. Examples of additional useful taxoids, including analogs of paclitaxel are taught by U.S. Pat. Nos. 6,610,860 to Holton, 5,530,020 to Gurram et al. and 5,912,264 to Wittman et al. Each of these patents is incorporated herein by reference.

Suitable DNA and/or RNA transcription regulators for use with the disclosed compounds include, without limitation, actinomycin D, daunorubicin, doxorubicin and derivatives and analogs thereof also are suitable for use in combination with the presently disclosed compounds.

DNA intercalators, cross-linking agents and alkylating agents that can be used in combination therapy with the disclosed compounds include, without limitation, cisplatin, carboplatin, oxaliplatin, mitomycins, such as mitomycin C, bleomycin, chlorambucil, cyclophosphamide, isophosphoramide mustard and derivatives and analogs thereof.

DNA synthesis inhibitors suitable for use as therapeutic agents include, without limitation, methotrexate, 5-fluoro-5′-deoxyuridine, 5-fluorouracil and analogs thereof.

Examples of suitable enzyme inhibitors for use in combination with the presently disclosed compounds include, without limitation, camptothecin, etoposide, formestane, trichostatin and derivatives and analogs thereof.

Suitable therapeutics for use with the presently disclosed compounds that affect gene regulation include agents that result in increased or decreased expression of one or more genes, such as, without limitation, raloxifene, 5-azacytidine, 5-aza-2′-deoxycytidine, tamoxifen, 4-hydroxytamoxifen, mifepristone and derivatives and analogs thereof.

The term “angiogenesis inhibitor” is used herein, to mean a molecule including, but not limited to, biomolecules, such as peptides, proteins, enzymes, polysaccharides, oligonucleotides, DNA, RNA, recombinant vectors, and small molecules that function to inhibit blood vessel growth. Angiogenesis inhibitors are known in the art and examples of suitable angiogenesis inhibitors include, without limitation, angiostatin K1-3, staurosporine, genistein, fumagillin, medroxyprogesterone, SFTI-1, suramin, interferon-alpha, metalloproteinase inhibitors, platelet factor 4, somatostatin, thromobospondin, endostatin, thalidomide, and derivatives and analogs thereof.

Other therapeutic agents, particularly anti-tumor agents, that may or may not fall under one or more of the classifications above, also are suitable for administration in combination with the presently disclosed compounds. By way of example, such agents include adriamycin, apigenin, erlotinib, gefitinib, temozolomide, rapamycin, topotecan, carmustine, melphalan, mitoxantrone, irinotecanetoposide, tenoposide, zebularine, cimetidine, and derivatives and analogs thereof.

The compounds disclosed herein may be administered orally, topically, transdermally, parenterally, via inhalation or spray and may be administered in dosage unit formulations containing conventional non-toxic pharmaceutically acceptable carriers, adjuvants and vehicles.

Typically, oral administration or administration via implantation or intravenously, such as via injection is preferred. However the particular mode of administration employed may be dependent upon the particular disease, condition of patient, toxicity of compound and other factors as will be recognized by a person of ordinary skill in the art.

In one embodiment, preferred therapeutic agents are identified herein by assessing their in vitro activity in a quantitative cell migration assay. In this assay, certain disclosed therapeutic agents exhibit in vitro IC₅₀ values against a model cell line of less than about 10 μM, such as from about 0.1 nM to about 1 μM, in particular from about 1 nM or 5 nM to about 500 nM, such as from about 50 nM to about 200 nM.

Suitable cell lines against which the disclosed compounds may be assessed are well known to those of skill in the art and include, by way of example, 4T1 breast cancer cells. Active compounds also can be identified and evaluated using a semiquantitative wound healing assay.

### EXAMPLES

The following examples are intended to be illustrative rather than limiting.

### General Methods

Part 1. Biological Assays

Antimicrobial Assays Compounds were tested for antimicrobial activity against Escherichia coli (ATCC 8739) and Bacillus subtilis (ATCC 49343) and for antifungal activity against Candida albicans (ATCC 90027) using a modified disc diffusion assay. Briefly, agar plates seeded with suspensions of bacteria or fungi were prepared by adding 500 μL of a 24 h culture of bacteria to 100 mL of autoclaved Antibiotic Medium 2 (AB2) containing 1% agar and cooled to 55° C., or of fungi to Sabouraud Dextrose Agar (SDA) at 55° C. Seeded liquid agar (10 mL) was transferred immediately to square Petri dishes and allowed to cool for 1 h. Controls used for C. albicans, E. coli and B. subtilis included amphotericin B (25 μg), ampicillin (50 μg) and choloramphenicol (12.5 μg), respectively. Sterile disks (6 mm) containing control antibiotics or one of the macrocycles (100 μg) were placed onto the agar and plates were incubated at 37° C. for 18 hours Inhibitory activity was measured by the zones of inhibition. Macrolides 1, 3 and 4 (50 μg/disk) inhibited B. subtilis with zones of inhibition of 9, 8.5 and 7.5 mm, respectively, in comparison to a 15 mm zone of inhibition by 50 μg of ampicillin. None of the macrolides tested measurably inhibited growth of E. coli and C. albicans.

In vitro Wound-Healing Assay: 4T1 breast tumor cells in medium containing 10% fetal bovine serum (FBS) were seeded into wells of 24-multiwell plates (Becton-Dickinson). After they grew to confluence, wounds were made with sterile pipette tips. Cells were washed with Phosphate Buffered Saline (PBS) and refreshed with medium with or without 10% FBS. After overnight incubation at 37° C., cells were fixed and photographed according to protocols described by Yang, S, and Huang, X.-Y. J. Biol. Chem. 2005, 280, 27130-27137; and Shan, D.; Chen, L.; Njardarson, J. T.; Gaul, C.; Ma, X.; Danishefsky, S. J.; and Huang, X.-Y. Proc. Natl. Acad. Sci. U.S.A. 2005, 102, 3772-3776, both the Yang and Shan references are incorporated herein by reference. The results of the wound healing assay demonstrate that macrolide 1 prevented closure of the wound by inhibiting cell migration.

Chamber Cell Migration Assay: Cell migration was assayed in Boyden chambers [8.0 μm pore size polyethylene terephthalate membrane, FALCON cell culture insert (Becton-Dickinson)]. Cells were trypsinized and counted. 5-10×10⁴ cells in serum free medium (300 μl) were added to the upper chamber and 500 μl of appropriate medium with 10% FBS were added to the lower chamber. Transwells were incubated for 4 h at 37° C. Cells on the inside of the transwell inserts were removed with a cotton swab, and cells on the underside of the insert were fixed and stained. Photographs of three random fields were taken and the number of cells counted to calculate the average number of cells that had transmigrated. Results in the form of IC₅₀ values obtained from the chamber migration assay using 4T1 breast tumor cells for compounds 1-6 are presented in Table 1.

TABLE 1

| Compound | IC₅₀ (nM) | |
|---|---|---|
| 1 | 77 ± 17 | nM |
| 2 | >250 | μM |
| 3 | 525 ± 163 | μM |
| 4 | 12 ± 2.9 | μM |
| 5 | >500 | μM |
| 6 | 550 ± 189 | nM |

Compounds 1-6, tested in Table 1, have the formulas:

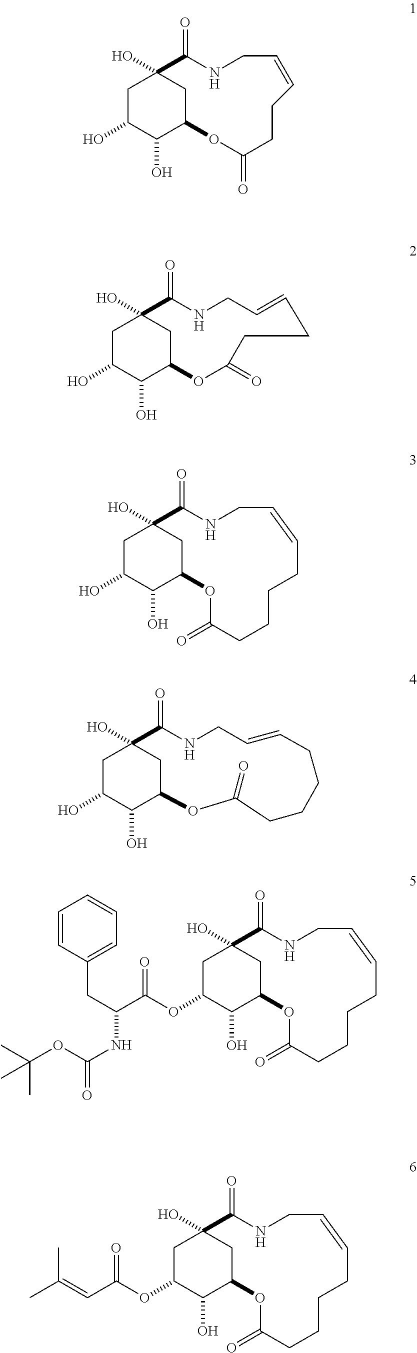

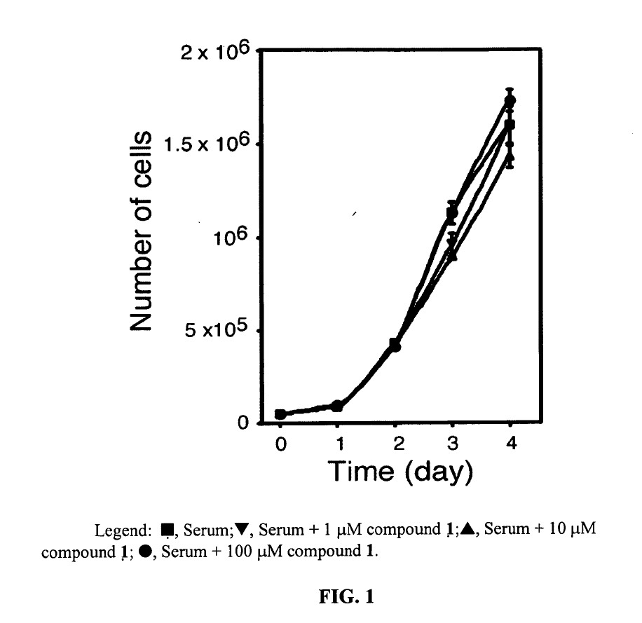

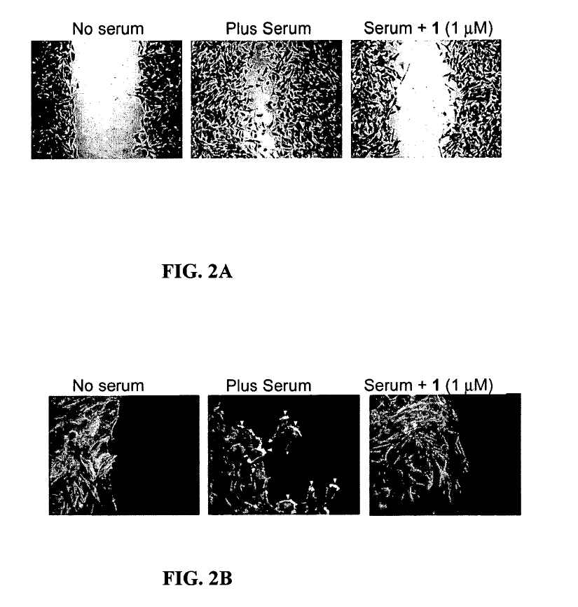

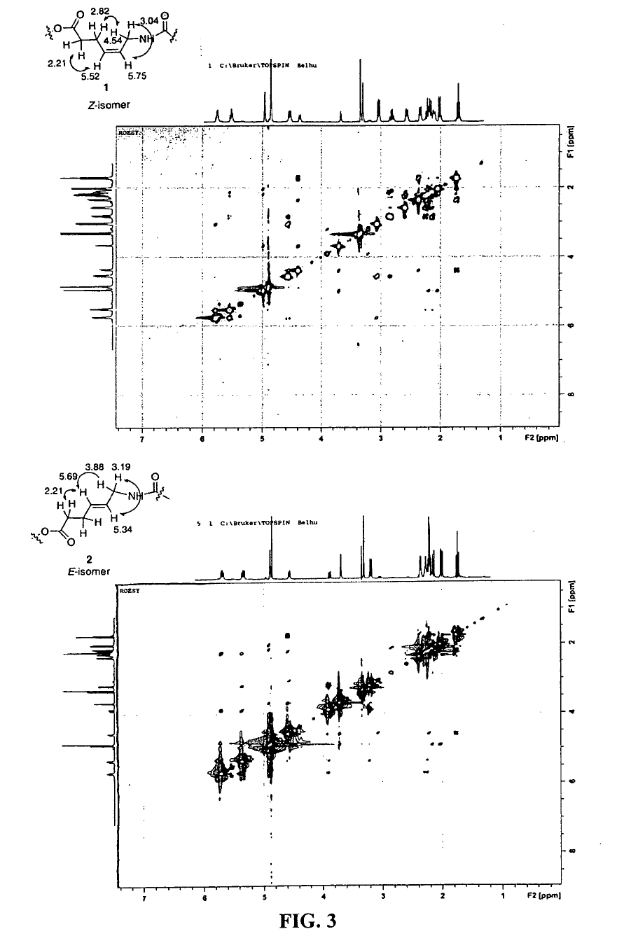

Cell Proliferation Assay: 4T1 breast tumor cells were trypsinized, resuspended in Dulbecco's Modified Eagle Medium (DMEM) with 10% FBS and counted. 5×10⁴ cells were plated per well in a 6-well dish and grown in DMEM with 10% FBS. Each day of the assay, one well of each type of cell was trypsinized and the cells were counted. The results of this assay are recorded in FIG. 1, which demonstrates that compound 1 does not affect 4T1 cell proliferation.

Fluorescence microscopy: Staining and observation of F-actin polymers were performed as previously described in Yang, S, and Huang, X.-Y. J. Biol. Chem. 2005, 280, 27130-27137; and Shan, D.; Chen, L.; Njardarson, J. T.; Gaul, C.; Ma, X.; Danishefsky, S. J.; and Huang, X.-Y. Proc. Natl. Acad. Sci. U.S.A. 2005, 102, 3772-3776. Briefly, cells were plated onto glass cover slips coated with gelatin. Cells were then fixed with 3.7% formaldehyde. The fixed cells were then permeabilized in 0.1% Triton X-100 for 5 min. After washing in PBS, phalloidin conjugated to rhodamine (Molecular Probes) in a solution containing PBS and 1% BSA was added to stain actin. After incubation for 30 min at room temperature, the cells were washed extensively to reduce nonspecific interactions. The cover slips were then fixed onto slides and imaged using a Zeiss fluorescence microscope.

### Part 2. Synthetic Procedures and Physical Data.

General experimental methods: All chemicals were purchased from Aldrich Chemical Co. and used without further purification unless otherwise specified. Thin layer chromatography (TLC) was performed using Merck KGaA Si gel 60 F₂₅₄ on aluminum plates (EMD Chemicals Inc.). TLCs were visualized by immersing the plates in 20% (w/v) phosphomolybdic acid solution in ethanol and developing over medium heat on a hot plate. Free amine-containing compounds were visualized with a ninhydrin spray purchased from Pierce Chemical Co. Compounds were purified by flash column chromatography (Si gel, Biotage), preparative TLC (20×20 cm Si gel GF plates, UV₂₅₄, ANALTECH), or analytical HPLC using a reverse-phase C-18 column (7 μM pore size, 7.8×300 mm). ¹H and ¹³C NMR spectra were recorded on Varian Mercury 300 or Bruker DMX500 or Bruker Avance 600 MHz spectrometers. Optical rotations were measured on a Perkin-Elmer Model 341 polarimeter. Low and high resolution mass spectra were obtained on a Waters LCT Premier mass spectrometer by electrospray (ES) or chemical ionization (APCI). IR spectra were recorded using a Perkin-Elmer Spectrum One FT-IR spectrophotometer.

### Example 1

### Synthesis of Cell Migration Inhibitor 1

This example describes the preparation of compound 1 via the synthetic scheme set forth below

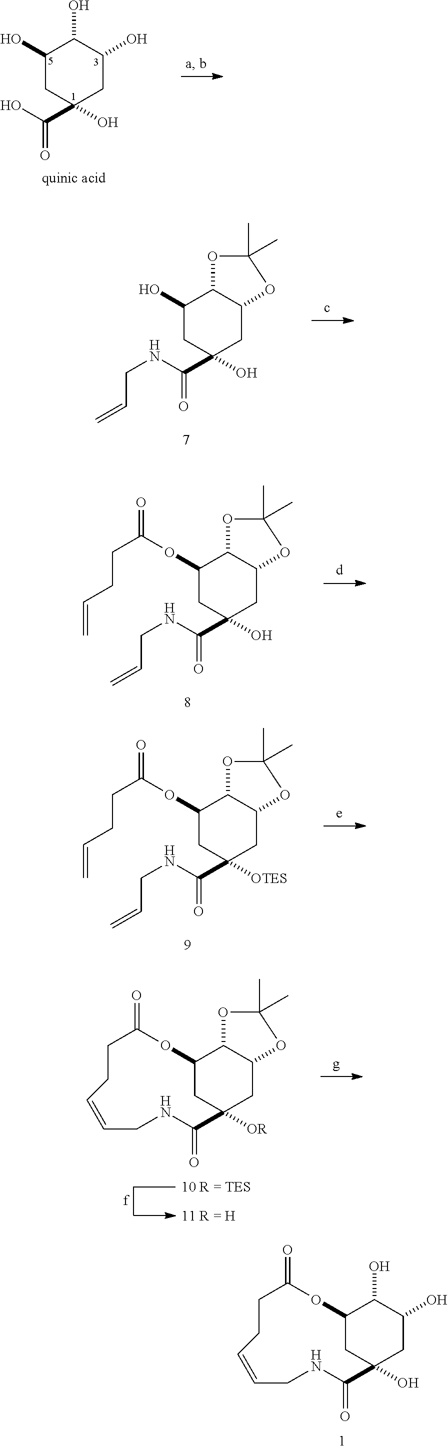

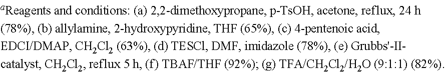

Compound 8, to a stirred solution of 4-pentenoic acid (288 mg, 2.88 mmol) in 10 mL of CH₂Cl₂ was added EDCI (598 mg, 3.12 mmol) and DMAP (80 mg, 0.65 mmol). After stirring for five minutes alcohol 7 (630 mg, 2.4 mmol) was added and the reaction was allowed to proceed until complete (4 h) as monitored by TLC (40% EtOAc/hexanes). The mixture was diluted with dichloromethane, washed successively with water, a saturated solution of NaHCO₃, water and finally brine, dried over MgSO₄, concentrated under reduced pressure, and chromatographed (Si gel, gradient elution 0-35% EtOAc/hexanes) to give 534 mg of 8 as a clear oil (63% yield). ¹H NMR (300 MHz, CDCl₃) δ 1.34 (s, 3H), 1.55 (s, 3H), 1.99 (m, 2H), 2.16 (dt, J=2.1, 12 Hz, 1H), 2.39 (m, 5H), 3.85 (t, J=1.5, 7.5 Hz, 2H), 4.11 (dd, J=5.4, 2.1, 1H), 4.50 (m, 1H), 5.05 (m, 4H), 5.29 (m, 1H), 5.79 (m, 2H), 7.14 (t, J=5.7 Hz, 1H); ¹³C NMR (75 MHz, CDCl₃) δ 26.1, 28.3, 29.0, 33.8, 34.1, 36.9, 41.6, 71.2, 74.94, 75.5, 77.0, 109.8, 115.7, 116.4, 134.0, 136.7, 172.5, 173.6; HRMS (APCI⁺) m/z 354.1922 MH⁺ (354.1917 Calcd for C₁₈H₂₈NO₆ MH⁺).

Compound 9: Imidazole (60 mg, 0.89 mmol) and triethyl chlorosilane (134 mg, 89 mmol) were added to a solution of 8 (210 mg, 0.59 mmol) in 2 mL of DMF, and this solution was maintained at room temperature for 2 h until the reaction had gone to completion as indicated by TLC (30% EtOAc/hexanes). The mixture was diluted with ethyl acetate and washed with water and then brine, concentrated under reduced pressure and chromatographed (Si gel, 10-25% EtOAc/hexanes) to give 215 mg of 9 as a clear oil (78% yield). ¹H NMR (300 MHz, CDCl₃) δ 0.72 (m, 6H), 0.97 (t, J=7.5 Hz, 6H), 1.32 (s, 3H), 1.47 (s, 3H), 1.85 (dd, J=5.1, 8.4, Hz, 1H), 2.02 (m, 2H), 2.29-2.46 (m, 5H), 3.83 (tt, J=1.5, 7.5 Hz, 2H), 4.22 (t, J=8.1 Hz, 1H), 4.57 (m, 1H), 4.95-5.00 (m, 1H), 5.06-5.20 (m, 4H), 5.80 (m, 2H), 6.73 (t, J=6.0 Hz, 1H); ¹³C NMR (75 MHz, CDCl₃) δ 6.4 (3C), 7.2 (3C), 25.1, 27.7, 29.0, 33.8, 38.1, 38.5, 41.8, 72.1, 72.5, 76.8, 77.1, 109.2, 115.7, 116.8, 133.9, 136.7, 172.6, 175.8; HRMS (APCI⁺) m/z 468.2783 MH⁺ (468.2781 Calcd for C₂₄H₄₂NO₆Si MH⁺).

Compound 10: Silyl ether 9 (250 mg, 0.53 mmol) was dissolved in dichloromethane (250 mL) and refluxed under 1 atm N₂. Grubbs'-II-catalyst (100 mg, 0.12 mmol) in 10 mL of dichloromethane was added via a syringe pump over a 2 h period and the mixture was maintained at reflux for 5 h. At this time, analysis of the mixture by LC-MS showed exclusively a molecular ion at 440 [M+H]⁺ corresponding to the desired macrocycle 10. The crude reaction mixture was concentrated under reduced pressure, dissolved in ethyl acetate treated with charcoal and passed through a pad of Celite® (Aldrich). The filtrate was concentrated under reduced pressure and chromatographed (Si gel, 5-25% EtOAc/hexanes) to give 162 mg of the desired macrocycles in 70% yield as a 5:1 ratio of Z/E isomers. 10(Z): R_{f}=0.39 (20% EtOAc/hexanes); [α]_{D}²⁰ +53.5 (c, 1.7 MeOH); ¹H NMR (300 MHz, CDCl₃) δ 0.59 (m, —SiCH₂, 6H), 0.91 (t, J=8.1 Hz, —CH₃, 9H), 1.31 (s, CH₃ 3H), 1.48 (s, CH₃, 3H), 1.52 (m, —CH₂, 1H), 2.01 (m, —CH₂, 2H), 2.10 (m, —CH₂, 1H), 2.22 (dd, J=3.0, 10.2 Hz, —CH₂, 1H), 2.56 (m, —CH₂, 2H), 2.89 (m, —CH₂, 1H), 2.99 (dd, J=5.1, 5.7 Hz, -═CHCH₂NHC═O, 1H), 3.96 (d, J=3.3 Hz, —CHOH, 1H), 4.67 (m, -═CHCH₂NHC═O, 1H), 4.98 (m, CHOH, 1H), 5.27 (m, CHOH, 1H), 5.54 (dt, J=3.3, 8.7 Hz, —CH═CH-1H), 5.66 (m, —CH═CH-1H), 5.97 (d, J=10.8 Hz, —CONH—, 1H); ¹³C NMR (75 MHz, CDCl₃) δ 6.5 (3C), 7.1 (3C), 24.3, 26.7, 28.7, 33.4, 35.4, 38.8, 39.4, 69.4, 73.7, 73.8, 76.2, 108.7, 126.8, 135.0, 171.3, 173.2; (cm⁻¹) 3439, 2949, 1738, 1670, 1504, 1246, 1108; HRMS (APCI⁺) m/z 440.2468 MH⁺ (440.2468 Calcd for C₂₂H₃₈NO₆Si MH⁺); 10(E) R_{f}=0.20 (20% EtOAc/hexanes); [α]_{D}²⁰ +36.0 (c, 0.8 MeOH); ¹H NMR (300 MHz, CDCl₃) δ 0.62 (q, J=7.8 Hz, —SiCH₂, 6H), 0.94 (t, J=8.1 Hz, —CH₃, 9H), 1.34 (s, —CH₃, 3H), 1.50 (s, —CH₃, 3H), 1.59 (dd, J=2.7, 9.6 Hz, —CH₂, 1H), 1.97 (dd, J=5.1, 10.2 Hz, —CH₂, 1H), 2.12 (m, —CH₂, 1H), 2.31 (m, —CH₂, 2H), 2.40 (m, —CH₂, 1H), 2.47 (m, —CH₂, 1H), 3.20 (dt, J=4.2, 5.4 Hz, -═CHCH₂NHC═O, 1H), 4.05 (m, CHOH 1H, -═CHCH₂NHC═O, 1H), 5.04 (m, CHOH, 1H), 5.23 (m, CHOH, 1H), 5.36 (m, —CH═CH—, 1H), 5.70 (m, —CH═CH—, 1H), 6.51 (m, —CONH—, 1H); ¹³C NMR (75 MHz, CDCl₃) δ 6.7 (3C), 7.2 (3C), 26.6, 28.7, 29.5, 36.1, 38.5, 39.0, 40.2, 70.2, 73.8, 74.2, 108.4, 126.7, 132.6, 173.0, 175.1; IR (cm⁻¹) 3431, 2950, 1732, 1677, 1493, 1243, 1155.; HRMS (APCI⁺) m/z 440.2474 MH⁺ (440.2468 Calcd for C₂₂H₃₈NO₆Si MH⁺).

Compound 11: To 10(Z) (25 mg, 0.06 mmol) was added TBAF in tetrahydrofuran (0.6 mL, 0.6 mmol). After stirring at room temperature for 2 h, the solvent was removed under reduced pressure. The residue was dissolved in ethyl acetate, washed with a saturated solution of NH₄Cl, water and then brine, and dried over sodium sulfate, concentrated under reduced pressure and chromatographed (Si gel, 20-40% EtOAc/hexanes) to give 18 mg of 11 (92% yield). ¹H NMR (300 MHz, CDCl₃) δ 1.34 (s, —CH₃, 3H), 1.51 (s, —CH₃, 3H), 1.70 (dd, J=4.8, 8.4 Hz, —CH₂, 1H), 2.10 (m, —CH₂, 3H), 2.40 (m, —CH₂, 2H), 2.63 (m, —CH₂, 1H), 2.81 (m, —CH₂, 1H), 2.96 (br, —OH, 1H) 3.25 (m, -═CHCH₂NHC=0, 1H), 4.03 (d, J=5.4 Hz, CHOH, 1H), 4.46 (m, -═CHCH₂NHC═O, 1H), 4.89 (m, —CHOH, 1H), 5.28 (m, —CHOH, 1H), 5.56 (dt, J=3.6, 7.8 Hz, —CH═CH—, 1H), 5.72 (m, —CH═CH— 1H), 6.37 (d, J=7.8 Hz, —CONH, 1H); ¹³C NMR (75 MHz, CDCl₃) δ 26.1, 28.3, 29.9, 34.1, 35.2, 36.1, 38.9, 69.6, 72.9, 73.2, 73.7, 109.0, 126.7, 134.8, 171.0, 173.7; HRMS (APCI⁺) m/z 326.1597 MH⁺ (326.1604 Calcd for C₁₆H₂₄NO₆ MH⁺).

Compound 1: To compound 11 (12 mg, 0.037 mmol) was added an ice cold mixture of TFA/CH₂Cl₂/H₂O (9:1:1, 1 mL) and this mixture was stirred for 15 min at room temperature. The mixture was dried under vacuum, co-evaporated with MeOH three times, and the residue was chromatographed by HPLC (gradient elution, 5-35% CH₃CN in water in 30 min) to give the desired macrolide 1 in 82% yield (8.6 mg). [α]_{D}²⁰ +67 (c, 1.1 MeOH); ¹H NMR (600 MHz, CD₃OD) δ 1.71 (t, J=12.6 Hz, —CH₂, 1H), 2.01 (dd, J=3.2, 11.6 Hz, —CH₂, 1H), 2.11 (m, —CH₂CH═CH—, 1H), 2.18 (dt, J=2.5, 14.9 Hz, —CH₂CH₂, 1H), 2.21 (dt, J=3.3, 10.0 Hz, —CH₂CH₂, 1H), 2.35 (m, —CH₂, 1H), 2.57 (m, J=2.8, 3.5, 4.9 Hz, —CH₂, 1H), 2.82 (m, —CH₂CH═, 1H), 3.04 (dd, J=6.7, 7.1, Hz, —CONHCH₂CH═, 1H), 3.35 (br, —OH, 3H) 3.68 (t, J=3.1 Hz, —CHOH, 1H), 4.37 (m, —CHOH, 1H), 4.54 (dd, J=5.0, 8.7 Hz, —CONHCH₂CH═, 1H), 4.96 (dt, J=2.9, 3.5 Hz, —CHOH, 1H); 5.52 (t, J=8.4 Hz, —CH═CH— 1H), 5.75 (dt, J=7.4, 8.2 Hz, —CH═CH—, 1H); ¹³C NMR (75 MHz, CD₃OD) δ 25.3, 34.6, 35.9, 36.2, 38.7, 67.6, 69.9, 73.7, 74.2, 128.3, 135.7, 173.2, 175.3; IR (cm⁻¹) 3330 (broad), 2934, 2855, 1711, 1648, 1528, 1450, 1256; HRMS (ES⁺) m/z 286.1293 MH⁺ (286.1291 Calcd for C₁₃H₂₀NO₆ MH⁺).

### Example 2

### Synthesis of Cell Migration Inhibitor 2

This example describes the synthesis of macrocycle 2 via the following scheme

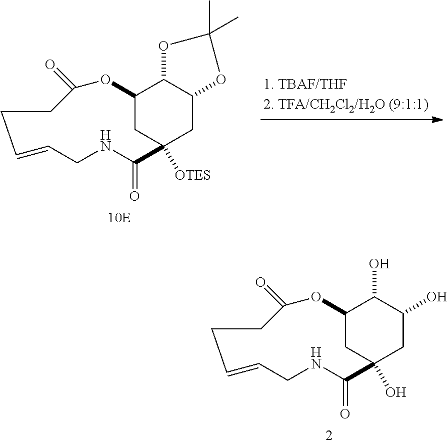

Compound 2. To 10(E) (10 mg, 0.023 mmol) (prepared as set forth in Example 1 above), was added TBAF in tetrahydrofuran (0.32 mL, 0.32 mmol). After stirring at room temperature for 2 h, the solvent was removed under reduced pressure. The residue was dissolved in ethyl acetate and washed with a saturated solution of NH₄Cl, water and then brine. After drying the organic layer over sodium sulfate and concentrating under reduced pressure, 1 mL of an ice cold solution of TFA/CH₂Cl₂/H₂O (9:1:1) was added to the residue and the mixture was allowed to stir for 15 min at room temperature. The solution was dried under vacuum, co-evaporated with MeOH three times, and then purified by HPLC (gradient elution: 5-35% CH₃CN in water in 30 min) to give the desired macrolide 2 in 61% yield (4.0 mg) for the two steps. [α]_{D}²⁰ +36.0 (c, 0.9 MeOH); ¹H NMR (600 MHz, CD₃OD) δ 1.74 (t, J=12.3 Hz, —CH₂, 1H), 2.00 (dd, J=3.7, 11.4 Hz, —CH₂, 1H), 2.14 (m, —CH₂, 1H), 2.15 (m, —CH₂, 1H), 2.18 (m, —CH₂—, 1H), 2.21 (m, 2H), 2.36 (m, 1H), 3.19 (dd, J=4.4, 9.7 Hz, —C(═O)—NH—CH₂CH═, 1H), 3.35 (bs, 3-OH), 3.69 (t, J=3.1 Hz, —CHOH—, 1H), 3.88 (dd, J=4.4, 9.7 Hz, —C(═O)—NH—CH₂CH═, 1H), 4.57 (m, —CHOH, 1H), 4.89 (dt, J=3.0, 3.1 Hz, —CHOH, 1H), 5.34 (m, —HC═CH—, 1H), 5.69 (m, —HC═CH—, 1H); ¹³C NMR (75 MHz, CD₃OD) δ 30.3, 36.2, 36.9, 38.6, 41.1, 67.7, 70.2, 74.5, 74.7, 128.2, 133.6, 174.8, 178.3; IR (cm⁻¹) 3278 (broad), 2933, 2470, 1700, 1646, 1462, 1345, 1247; HRMS (ES⁺) m/z 286.1299 MH⁺ (286.1291 Calcd for C₁₃H₂₀NO₆ MH⁺).

### Example 3

### Synthesis of Cell Migration Inhibitors 3 and 4

This example describes the synthesis of macrocyclic compounds 3 and 4 according the scheme:

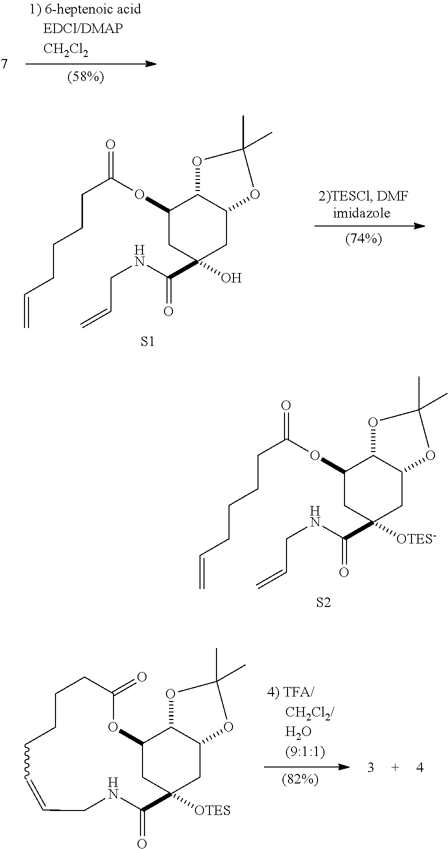

General procedure for synthesis of 3 and 4: As illustrated above, compounds 3 and 4 were synthesized and purified using the same conditions as described in detail above for macrocycles 1 and 2, with the exception that 7 was esterified with 6-heptenoic acid instead of 4-pentenoic acid, and the mixture of Z and E isomers that were the products of the macrocyclization were globally deprotected with TFA and chromatographed by RP-HPLC (gradient elution: 5-35% CH₃CN in water in 30 min).

Compound S1: ¹H NMR (300 MHz, CDCl₃) δ 1.35 (s, 3H), 1.40 (m, 2H), 1.58 (s, 3H), 2.10 (m, 4H), 1.96 (m, 1H), 2.35 (m, 2H), 2.42 (dd, J=3.4, 10.2 Hz, 1H), 3.83 (m, 3H), 4.16 (m, 1H), 4.57 (m, 1H), 4.98 (m, 2H), 5.16 (m, 2H), 5.31 (m, 1H), 5.80 (m, 2H), 7.16 (t, J=3 Hz, 1H); ¹³C NMR (75 MHz, CD₃OD) δ 24.5, 26.2, 28.3, 28.4, 33.5, 34.1, 34.4, 37.0, 41.7, 71.0, 75.0, 75.6, 77.1, 109.8, 114.9, 116.5, 134.0, 138.6, 173.2, 173.6.

Compound S2: ¹H NMR (300 MHz, CDCl₃) δ 0.73 (m, 6H), 0.98 (t, J=8.1 Hz, 9H), 1.33 (s, 3H), 1.41 (m, 1H), 1.48 (s, 3H), 1.62 (m, 3H), 1.87 (dd, J=4.5, 9 Hz, 1H), 2.05 (m, 4H), 2.34 (m, 3H), 3.85 (t, J=5.7 Hz, 2H), 4.23 (t, J=7.8 Hz, 1H), 4.58 (dt, J=8.4 Hz, 1H), 4.96 (m, 2H), 5.14 (m, 3H), 5.80 (m, 2H), 6.74 (t, J=3 Hz, 1H), ¹³C NMR (75 MHz, CDCl₃) δ 6.42 (3C), 7.2 (3C), 24.6, 25.1, 27.7, 28.5, 33.5, 34.5, 38.1, 38.6, 41.8, 72.0, 72.5, 76.8, 76.9, 109.2, 114.9, 116.8, 133.9, 138.7, 173.3, 175.8; HRMS (ES⁺) m/z 496.3071 MH⁺ (496.3094 Calcd for C₂₆H₄₆NO₆Si MH⁺).

Compound 3: [α]_{D}²⁰ +50.0 (c, 1.1 MeOH) ¹H NMR (600 MHz, CD₃OD) δ 1.32 (m, —CH₂, 1H), 1.49 (m, —CH₂, 1H), 1.62 (m, —CH₂, 2H), 1.78 (t, J=12.1 Hz, —CH₂, 1H), 1.96 (m, —CH₂, 1H), 2.00 (dd, J=3.4, 11.3 Hz, —CH₂, 1H), 2.15 (dt, J=2.3, 10.1 Hz, —CH₂, 1H), 2.23 (ddd, J=1.4, 6.9 Hz, —CH₂, 2H), 2.28 (m, —CH₂, 1H), 2.38 (m, —CH₂, 1H), 3.40 (dd, J=6.5, 7.3 Hz, -═CHCH₂NHCO—, 1H), 3.72 (m, CHOH, 1H), 4.29 (m, —CHOH 1H, -═CHCH₂NHCO—, 1H), 5.02 (dt, J=3.4, 3.2 Hz, CHOH, 1H) 5.55 (dt, J=4.6, 5.9 Hz, —CH═CH—, 1H), 5.68 (dt, J=3.2, 6.5, 7.1 Hz, —CH═CH— 1H); ¹³C NMR (75 MHz, CD₃OD) δ 23.9, 26.4, 31.3, 33.8, 36.3, 38.6, 42.1, 67.6, 69.3, 73.2, 74.7, 128.9, 133.5, 174.7, 177.1; IR (cm⁻¹) 3348 (broad), 2923, 2479, 1693, 1652, 1543, 1460, 1149; HRMS (ES⁺) m/z 314.1616 MH⁺ (314.1604 Calcd for C₁₅H₂₄NO₆ MH).

Compound 4: [α]_{D}²⁰ +19.0 (c, 1.8 MeOH) ¹H NMR (300 MHz, CD₃OD) δ 1.49 (m, —CH₂, 1H), 1.58 (m, —CH₂, 1H), 1.72 (m, CH₂, 2H), 1.77 (t, J=12.7 Hz, —CH₂, 1H), 2.05 (dd, J=3.5, 11.2 Hz, —CH₂, 1H), 2.10 (m, —CH₂, 2H), 2.16 (m, —CH₂, 2H), 2.19 (t, J=7.9 Hz, —CH₂, 1H), 2.20 (m, —CH₂, 1H), 3.42 (dd, J=5.0, 10.1 Hz, -═CHCH₂NHCO—, 1H), 3.70 (t, J=3.2 Hz, —CHOH, 1H), 3.90 (dd, J=7.0, 7.4 Hz, -═CHCH₂NHCO—, 1H), 4.26 (dt, J=3.3, 4.4 Hz, —CHOH, 1H), 4.99 (dt, J=3.0, 3.3 Hz, —CHOH, 1H), 5.46-5.52 (m, —CH═CH—, 2H), 8.0 (—CONH); ¹³C NMR (75 MHz, CD₃OD) δ 24.2, 26.3, 27.8, 33.4, 36.0, 36.9, 38.7, 67.6, 69.5, 73.1, 74.4, 126.1, 135.3, 174.4, 176.5; IR (cm⁻¹) 3350, 2927, 1721, 1639, 1524, 1454, 1138; HRMS (ES⁺) m/z 314.1599 MH⁺ (314.1604 Calcd for C₁₅H₂₄NO₆ MH⁺).

### Example 4

### Procedure for the Synthesis of Compound 5

This example describes the preparation of compound 5 via a regioselective esterification reaction: N-(tert-butoxycarbonyl)-L-phenylalanine (2.2 mg 8.1 μmol), EDCI (1.7 mg, 9.0 μmol) and DMAP (1 mg, 8.2 μmol) were dissolved in 1 mL of dichloromethane and stirred for 5 min, at which point 3 (2.3 mg, 7.4 μmol) was added and the mixture was stirred at room temperature for 2 h. The solution was diluted with dichloromethane (8 mL) and washed with water, saturated sodium bicarbonate and washed again with water and brine and dried over MgSO₄ and concentrated under reduced pressure. The crude product was purified by RP-HPLC (gradient elution: 5-65% CH₃CN in water in 60 min) to give compound 5 (3.6 mg) in 87% yield. [α]_{D}²⁰ +22.0 (c, 1.7 MeOH) ¹H NMR (300 MHz, CD₃OD) δ 1.31 (m, —CH₂, 1H), 1.37 (s, —C(CH₃)₃, 9H), 1.46 (m, —CH₂, 2H), 1.59 (m, —CH₂, 1H), 1.76 (t, J=12 Hz, —CH₂, 1H), 2.03 (dd, J=3.3, 8.4 Hz, —CH₂, 1H), 2.09-2.16 (m, —CH₂, 3H), 2.19 (t, J=8.1 Hz, —CH₂, 1H), 2.25 (m, —CH₂, 1H), 2.88 (dd, J=5.1, 8.7 Hz, PhCH₂—, 1H), 3.08 (dd, J=5.4, 8.4 Hz, PhCH₂—, 1H), 3.40 (m, -═CHCH₂NHCO—, 1H), 3.70 (t, J=3.3 Hz, —CHOH— 1H), 3.89 (dd, J=6.3, 8.1 Hz, -═CHCH₂NHCO—, 1H), 4.26 (m, -t-BocNHCHCOO—, 1H), 4.34 (m, —CHOH, 1H), 4.99 (m, —CHOH, 1H), 5.45-5.52 (m, CH═CH, 2H), 7.18-7.26 (m, ArH, 5H); ¹³C NMR (75 MHz, CDCl₃) δ 23.9, 26.4, 28.8 (3C), 31.3, 33.8, 36.3, 38.6, 38.9, 42.0, 57.5, 67.6, 69.4, 73.3, 74.7, 127.9, 128.9, 129.6, 130.4, 133.5, 135.3, 174.4, 174.7, 181.3, 182.5; IR (cm⁻¹) 3348, 2927, 2509, 1713, 1649, 1513, 1365; 1225; HRMS (ES⁺) m/z 583.2612 MNa⁺ (583.2632 Calcd for C₂₉H₄₀N₂O₉ MNa⁺).

### Example 5

### Procedure for the Synthesis of Compound 6

This example describes the synthesis of compound 6 via a regioselective esterification reaction: To a solution of 3-methyl-2-butenoic acid (7.4 mg, 7.4 μmol), EDCI (1.7 mg, 9.0 μmol) and DMAP (1.0 mg, 8.2 μmol) in dichloromethane (1 mL) was added 3 (2.1 mg, 6.7 mmol). The mixture was stirred at room temperature for 2 h, and then diluted with dichloromethane (8 mL) and washed with water, saturated sodium bicarbonate, water and then brine. The organic layer was dried over MgSO₄ and concentrated under reduced pressure. The crude product was purified by RP-HPLC (gradient elution: 5-65% CH₃CN in water in 55 min) to give 5 (2.4 mg) in 91% yield. [α]_{D}²⁰ +34.0 (c, 1.7 MeOH); ¹H NMR (600 MHz, CD₃OD) δ 1.64 (m, —CH₂, 1H), 1.73 (m, —CH₂, 1H), 1.89 (m, —CH₂, 1H), 1.92 (s, —CH₃, 3H), 2.00 (t, J=13 Hz, —CH₂, 1H), 2.08 (dd, J=4.3, 5.1 Hz, —CH₂, 1H); 2.12 (m, —CH₂, 2H), 2.17 (s, —CH₃, 3H), 2.20-2.27 (m, —CH₂CH₂— 4H), 3.51 (dd, J=5.6, 9.1 Hz, -═CHCH₂NHCO—, 1H), 3.86 (dd, J=6.7, 9.1 Hz, -═CHCH₂NHCO—, 1H), 3.93 (t, J=3.2 Hz, CHOH, 1H), 4.99 (dt, J=5.3, 5.5 Hz, CHOH, 1H), 5.39 (m, —CHOH, 1H), 5.52 (m, —CH═CH—, 2H), 5.75 (m, —(CH₃)₂C═CH—, 1H); ¹³C NMR (75 MHz, CD₃OD) δ 22.9, 26.3, 28.8, 30.0, 33.6, 36.3, 37.8, 38.6, 41.5, 69.4, 73.2, 75.5, 76.9, 119.6, 131.3, 136.1, 161.1, 170.0, 177.2, 179.0; IR (cm⁻¹) 3365, 2929, 1673, 1644, 1540, 1440, 1202; HRMS (ES⁺) m/z 396.2010 MH⁺ (396.2022 Calcd for C₂₀H₃₀NO₇ MO.

### Example 6

### Inhibition of Tumor Metastasis in Mice

This example describes the inhibition of breast cancer metastasis in mice using the presently disclosed cell migration inhibitors, such as compound 1.

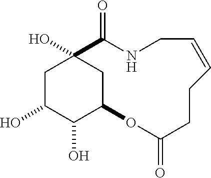

### Compound 1

4T1 mouse breast tumor cells are isolated from a single spontaneously arising mammary tumor from a BALB/BfC3H mouse (MMTV+) (Miller, F. R., Miller, B. E., and Heppner, G. H. Invasion Metastasis 1983, 33, 22-31). The 4T1 tumor closely mimics human breast cancer in its anatomical site, immunogenicity, growth characteristics, and metastatic properties. From the mammary gland, 4T1 tumor spontaneously metastasizes to a variety of target organs including the lung, bone, brain, and liver through primarily a hematogenous route (Aslakson and Miller Cancer Research, 1992, 52, 1399-1405.

To demonstrate the efficacy of therapeutic application of the disclosed macrocycles in the 4T1 murine mammary carcinoma models, the macrocycles can be administered to BALB/c mice carrying the 4T1 tumors.

Female BALB/c mice (6-8 week old) can be purchased from the Jackson Laboratory (Bar. Harbor, Me.). 4T1 tumor cells (1×10⁵) are injected subcutaneously into the abdominal mammary gland area of mice in 0.1 mL of a single-cell suspension in phosphate buffered saline (PBS) on Day 0. This dosage of tumor cells gives rise to tumors of about 10 mm in diameter in untreated wild type mice in 21-23 days. On Day 7, when the tumors average in size about 4-5 mm in diameter, a currently disclosed macrocycle, such as compound 1, or control PBS saline are administered daily by intraperitoneal injection at 10 mg/kg, 20 mg/kg or 50 mg/kg per mouse until Day 25. On Day 28, the mice are sacrificed. Primary tumors can be measured using electronic calipers on the day when the mice were sacrificed. Tumor size can be reported as the square root of the product of two perpendicular diameters. Numbers of metastatic 4T1 cells in lung can be determined by the clonogenic assay. In brief, lungs are removed from each mouse after sacrifice, finely minced and digested in 5 mL of enzyme cocktail containing 1PBS and 1 mg/mL collagenase type IV for 2 hours at 37° C. After incubation, samples are filtered through 70 μM nylon cell strainers and are washed twice with PBS. Resulting cells are suspended, plated and serially diluted in 10 cm tissue culture dishes in medium RPMI1640 containing 60 μM thioguanine for clonogenic growth. 6-Thioguanine-resistant tumor cells formed foci after 14 days, at which time they are fixed with methanol and stained with 0.03% methylene blue for counting.

### Example 7

### Treatment of a Subject Having Metastatic Breast Cancer

This example describes treatment of a subject having metastatic breast cancer by administering a cell migration inhibitor, such as compound 1, to the subject. A subject having metastatic breast cancer is selected for treatment and treated with a dosage regimen that may be tailored to the patient's conditions and response in a manner that is conventional for any therapy, and may need to be adjusted in response to changes in conditions. For example, the attending clinician, in judging whether treatment is effective at the dosage administered, will consider the general well-being of the patient as well as more definite signs such as relief of disease-related symptoms, inhibition of tumor growth, actual shrinkage of the tumor, or inhibition of metastasis. Size of the tumor can be measured by standard methods such as direct measurement or imaging studies, e.g., CAT or MRI scan, and successive measurements can be used to judge whether or not growth of the tumor has been retarded or even reversed. Relief of disease-related symptoms such as pain, and improvement in overall condition also can be evaluated to help judge effectiveness of treatment.

A dosage regimen may include administration of an oral formulation of compound 1, daily for 5 days every 3 weeks to adult patients at doses ranging from about 30 to about 90 mg/m² (based on body surface area) or daily for 14 days every 3 weeks at doses ranging from about 10 to about 50 mg/m² (based on body surface area). For example, an oral formulation of compound 1 may be administered daily for 5 days every 3 weeks to adult subjects at doses ranging from about 50 to about 70 mg/m² (based on body surface area) or daily for 14 days every 3 weeks at doses ranging from about 25 to about 35 mg/m² (based on body surface area). More typically, an oral formulation of compound 1 may be administered daily for 5 days every 3 weeks to adult patients at a dose of about 60 mg/m² or 70 mg/m² (based on body surface area) or daily for 14 days every 3 weeks at a dose of 30 mg/m² (based on body surface area).

The disclosed cell migration inhibitors can coadministered with another chemotherapeutic agent. Two exemplary regimens for use in combination with a cell migration inhibitor include: a) cyclophosphamide and doxorubicin or epirubicin, if patients have not received anthracycline therapy in the adjuvant setting, or b) paclitaxel, if patients have received any anthracycline therapy in the adjuvant setting.

Subjects having tumors characterized by overexpression of the ErbB2 (HER2) oncogene as determined by immunohistochemistry or fluorescence in situ hybridization (FISH), can be treated with an anti-HER2 antibody, such as trastuzumab, which is marketed under the trade name HERCEPTIN®, in combination with the disclosed cell migration inhibitors. Tumor expression of HER2 can be determined by immunohistochemical analysis of a set of thin sections prepared from the subject's paraffin-archived tumor blocks. The primary detecting antibody typically used is murine 4D5 MAb, which has the same CDRs as the humanized antibody used for the treatment. Tumors are considered to overexpress HER2 if at least about 25% of tumor cells exhibit characteristic membrane staining by anti-p185 HER2 antibody.

Cyclophosphamide (600 mg/m²) is given either by iv push over a minimum period of 3 minutes or by infusion over a maximum period of 2 hours.

Doxorubicin (60 m g/m²) or epirubicin (75 mg/m²) are given either by slow iv push over a minimum period of 3-5 minutes or by infusion over a maximum period of 2 hours, according to institutional protocol.

Paclitaxel is given at a dose of 175 mg/m² over 3 hours by intravenous administration.

The response to a treatment regimen can be assessed as is known to those skilled in the art of clinical oncology. For example the size of measurable cancerous lesions can be monitored and the progression, increase in size or even regression or disappearance such lesions can be recorded. Similarly the appearance of new measurable lesions can be monitored and the treatment regimen adjusted appropriately as is known to those of skill in the art.

In view of the many possible embodiments to which the principles of the disclosed invention may be applied, it should be recognized that the illustrated embodiments are only preferred examples of the invention and should not be taken as limiting the scope of the invention. Features, characteristics, compounds, chemical moieties, compositions and method for their use described in conjunction with a particular aspect, embodiment, or example of the invention are to be understood to be applicable to any other aspect, embodiment, or example of the invention. Accordingly, this disclosure includes all modifications encompassed within the spirit and scope of the disclosure as defined by the following claims.

1. A compound having the structure: 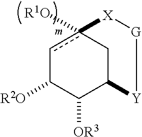
or any pharmaceutically acceptable salt or solvate thereof, wherein:
m is 0 or 1;
R¹, R² and R³ independently are H, aralkyl, acyl, lower alkyl or silyl;
X is —C(O)N(R⁴)— or —C(S)N(R⁴)—; —C(O)—; —C(S)—;
Y is —OC(O)—; —OC(O)N(R⁵)—; —N(R⁵)C(O)—; or —OC(O)O—;
G comprises a saturated or unsaturated aliphatic chain having from 2 to about 10 atoms in the chain, the chain optionally including 1, 2, or 3 heteroatoms; the chain optionally being substituted with 1, 2 or 3 substituents independently selected from lower alkyl, —OR⁶, epoxy, aziridinyl, cyclopropyl, —NR⁷R⁸ and halo;
R⁴, R⁵, R⁶, R⁷ and R⁸ independently are selected from H, lower alkyl and acyl.

2. The compound of claim 1, having the structure

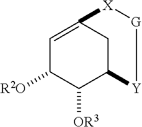

3. The compound of claim 1, having the structure

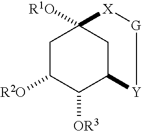

4. The compound claim 1, wherein G comprises an unsaturated aliphatic chain.

5. The compound of claim 1, wherein G comprises from 4 to 8 carbon atoms in the saturated or unsaturated aliphatic chain.

6. The compound of claim 1, having the structure

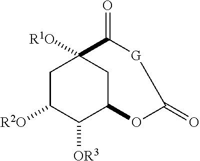

7. The compound of claim 1, having the structure

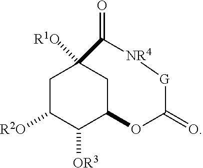

8. The compound of claim 1, wherein G comprises an alkenyl moiety in the aliphatic chain.

9. The compound of claim 6, wherein the alkenyl moiety is a Z alkene.

10. The compound of claim 1, wherein at least one of R¹, R² and R³ is an acyl group.

11. The compound of claim 1, having the structure 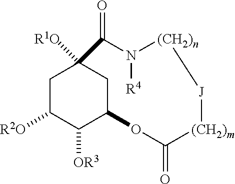
wherein n is an integer from 1 to 5; and
m is an integer from 0 to 5.

12. The compound of claim 11, wherein J has the structure 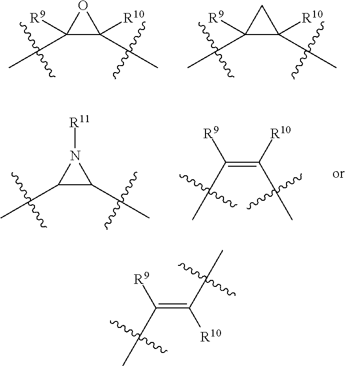
wherein R⁹ and R¹⁰ independently are H or optionally substituted lower alkyl; and R¹¹ is H, lower alkyl, acyl or sulfonyl.

13. The compound of claim of claim 12 wherein at least one of R⁹ and R¹⁰ is H.

14. The compound of claim 12, wherein G has the formula

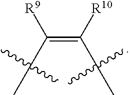

15. The compound of claim 14, wherein at least one of R⁹ and R¹⁰ is methyl.

16. The compound of claim 1, wherein the compound has the structure

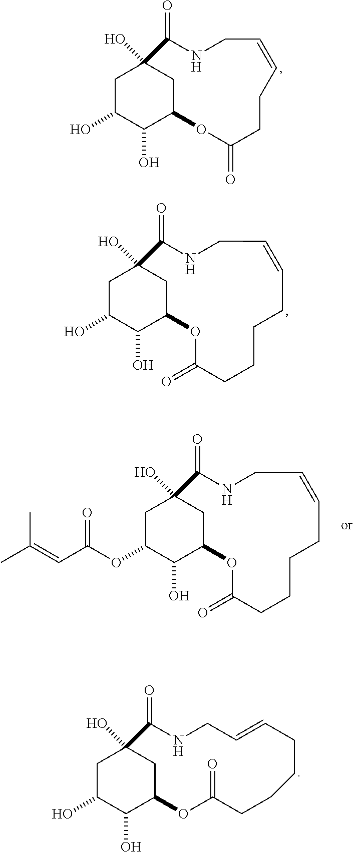

17. A method for making a compound of claim 1, comprising providing an intermediate compound of the structure 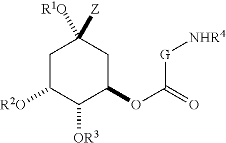
wherein Z is —C(O)OH or an activated ester moiety; and
performing a macrolactamization reaction to provide a compound of claim 1.

18. The method of claim 17, wherein the intermediate compound has the structure

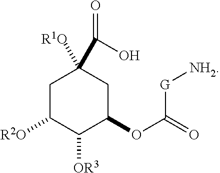

19. A method for making a compound of claim 1, comprising providing a compound having the structure 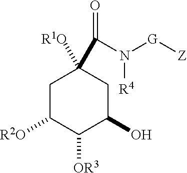
wherein Z is —C(O)OH or an activated ester moiety; and
performing a macrolactonization reaction to form a compound of claim 1.

20. The method of claim 19, wherein the intermediate compound has the structure

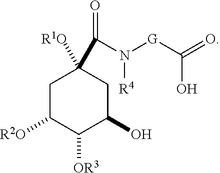

21. A method for making a compound of claim 12, comprising providing an intermediate compound of the formula 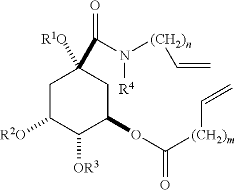
and performing a ring closing metathesis reaction.

22. The method of claim 20, wherein performing the ring closing metathesis reaction comprises contacting the intermediate compound with a metathesis catalyst.

23. The method of claim 22, wherein the catalyst is a ruthenium carbene-based catalyst.

24. The method of claim 22, wherein the catalyst is a molybdenum carbene-based catalyst.

25. A pharmaceutical composition, comprising:
a pharmaceutically acceptable, carrier, adjuvant or vehicle; and
an effective amount of a compound having the structure: 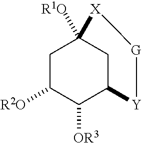
or any pharmaceutically acceptable salt thereof, wherein:
R¹, R² and R³ independently are H, aralkyl, acyl, lower alkyl or silyl;
X is —C(O)N(R⁴)— or —C(S)N(R⁴)—; —C(O)—; —C(S)—;
Y is —OC(O)—; —OC(O)N(R⁵)—; —N(R⁵)C(O)—; or —OC(O)O—;
G comprises a saturated or unsaturated aliphatic chain having from 2 to about 10 atoms in the chain, the chain optionally including 1, 2, or 3 heteroatoms; the chain optionally being substituted with 1, 2 or 3 substituents independently selected from lower alkyl, —OR⁶, epoxy, —NR⁷R⁸ and halo;
R⁴, R⁵, R⁶, R⁷ and R⁸ independently are selected from H, lower alkyl and acyl.

26-28. (canceled)

29. The composition of claim 25, further comprising a cytotoxic agent.

30. The composition of claim 29, wherein the cytotoxic agent is an anticancer agent.

31. The composition of claim 30, wherein the anticancer agent is selected from the microtubule binding agents, DNA intercalators, DNA alkylating agents, DNA cross-linkers, DNA synthesis inhibitors, DNA and/or RNA transcription inhibitors, enzyme inhibitors, gene regulators, enzymes, antibodies and angiogenesis inhibitors.

32. The composition of claim 30, wherein the anticancer agent is selected from erlotinib, gefitinib, temozolomide, paclitaxel, docetaxel, daunorubicin, cisplatin, carboplatin, oxaliplatin, colchicine, dolastatin 15, nocodazole podophyllotoxin, rhizoxin, vinblastine, vindesine, vinorelbine (navelbine), the epothilones, the mitomycins, bleomycin, chlorambucil, carmustine, melphalan, mitoxantrone, 5-fluoro-5′-deoxyuridine, camptothecin, SFTI-1, topotecan, irinotecanetoposide, tenoposide, geldanamycin, methotrexate, adriamycin, actinomycin D, medroxyprogesterone, mifepristone, raloxifene, 5-azacytidine, 5-aza-2′-deoxycytidine, zebularine, tamoxifen, 4-hydroxytamoxifen, apigenin, rapamycin, angiostatin K1-3, L-asparaginase, staurosporine, genistein, fumagillin, endostatin, isophosphoramide mustard, thalidomide and analogs thereof.

34-53. (canceled)

54. A method for treating a subject for a pathologic condition that responds to treatment with a cell migration inhibitor comprising decreasing or inhibiting cell migration by administering to the subject an effective amount of a compound according to claim 1.

55. The method according to claim 54, wherein the condition is characterized by neovascularization.

56. The method according to claim 54, wherein the pathologic condition is a metastatic cancer.

57. A method for treating a subject having a hyperproliferative disorder, comprising administering to the subject a therapeutically effective amount of a composition according to claim 25.

58. The method of claim 57, wherein the hyperproliferative disorder comprises brain cancer, breast cancer, bladder cancer, bone cancer, cervical cancer, colon cancer, central nervous system cancer, esophageal cancer, gall bladder cancer, gastrointestinal cancer, head and neck cancer, Hodgkin's Disease, non-Hodgkin's lymphomas, laryngeal cancer, leukemia, lung cancer, melanoma, neuroblastoma, ovarian cancer, pancreatic cancer, prostate cancer, rectal cancer, renal cancer, retinoblastoma, stomach cancer, testicular cancer, Wilms' tumor or a combination thereof.

59. The method of claim 57, wherein the method is used to inhibit the metastasis of prostate, breast, colon, bladder, cervical, skin, testicular, kidney, ovarian, stomach, brain, liver, pancreatic or esophageal cancer, or lymphoma, leukemia or multiple myeloma.

60. A method for treating or inhibiting the severity of tumor cell metastasis in a subject, comprising administering to a subject in need thereof a therapeutically effective amount of a composition according to claim 25.

61. A method for treating an angiogenesis-dependent disorder in a subject comprising administering to the subject an angiogenesis inhibiting amount of a composition of claim 25.

62. The method of claim 61, wherein the angiogenesis-dependent disorder is an ocular neovascular disease, myocardial angiogenesis, diabetic retinopathy, corneal graft rejection, Crohn's disease, rheumatoid arthritis or tumor metastasis.

63. The method of claim 61, wherein the angiogenesis-dependent disorder is a cardiovascular disorder.

64. The method of claim 63, wherein the cardiovascular disorder is polyarteritis, sickle cell anemia, atherosclerosis, artery occlusion, vein occlusion or combinations thereof.

65. The method of claim 54, wherein the effective amount of compound is from about 1 milligram to about 50 milligrams for each kilogram of the subject's body weight.

66. The method of claim 54, wherein the effective amount of compound is from about 0.1 milligram to about 40 milligrams for each kilogram of the subject's body weight.

67. The method of claim 54, wherein the effective amount of compound is from about 1 milligram to about 40 milligram for each kilogram of the subject's body weight.

68. The method of claim 54, wherein the effective amount of compound is 10 milligrams or more for each kilogram of the subject's body weight.

69. The method of claim 54, wherein the compound is 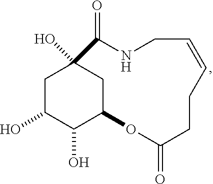
and the effective amount is at least 77±17 nM; or the compound is 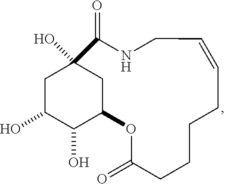
and the effective amount is at least 525±163 μM; or the compound is 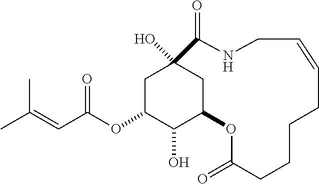
and the effective amount is at least 550±189 nM; or the compound is 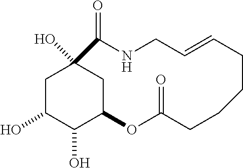
and the effective amount is at least 12±2.9 μM.
